(19) United States
(12) Patent Application Publication
(10) Pub. No.: US 2026/0041555 A1
(43) Pub. Date: Feb. 12, 2026

(54) HEART VALVE TREATMENT DEVICES AND DELIVERY DEVICES THEREFOR

(71) Applicant: EDWARDS LIFESCIENCES CORPORATION, Irvine, CA (US)

(72) Inventors: Sergio Delgado, Irvine, CA (US); Aviv Galon, Tel Aviv (IL); Itai Pelled, Ramat-Hasharon (IL); Meir Abucasis, Hadera (IL); Noam Nir, Pardes-Hanna (IL); Bar Eytan-Vaisman, Gedera (IL); Gil Hamerman, Kfar Yona (IL); Travis Zenyo Oba, Yorba Linda, CA (US)

(21) Appl. No.: 19/366,499

(22) Filed: Oct. 22, 2025

Related U.S. Application Data
(63) Continuation of application No. PCT/US2024/025959, filed on Apr. 24, 2024.
(60) Provisional application No. 63/462,183, filed on Apr. 26, 2023.
(60) Provisional application No. 63/613,675, filed on Dec. 21, 2023.

(51) Int. Cl. A61F 2/24

(52) U.S. Cl. CPC A61F 2/2463; A61F 2210/0004; A61F 2220/0008; A61F 2220/0075; A61F 2220/0083; A61F 2250/0001

(57) ABSTRACT

A valve treatment device having a coaptation element, a first anchor assembly configured to grasp a first leaflet of a native heart valve, and a second anchor assembly configured to grasp a second leaflet of the native heart valve. The first anchor assembly is connected to a first side of the coaptation element. The second anchor assembly is connected to a second side of the coaptation element. The valve treatment device can include a bioabsorbable central portion between the first anchor assembly and the second anchor assembly.

### CROSS REFERENCE TO RELATED APPLICATIONS

This application is a continuation of Patent Cooperation Treaty Application No. PCT/US2024/025959, filed on Apr. 24, 2024, which claims the benefit of U.S. Provisional Patent Application No. 63/613,675 filed Dec. 21, 2023 and titled “HEART VALVE TREATMENT DEVICES AND DELIVERY DEVICES THEREFOR” and claims the benefit of U.S. Provisional Patent Application No. 63/462,183 filed Apr. 26, 2023 and titled “HEART VALVE TREATMENT DEVICES AND DELIVERY DEVICES THEREFOR” to Delgado et al. The above applications are incorporated by reference herein in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for treating a native valve of a subject (e.g., a living subject, a simulation, etc.) are disclosed. The devices can be valve treatment devices, valve repair devices, implantable devices, valve treatment devices, implants, etc. While sometimes described as an implantable device for illustration purposes in various examples herein, similar configurations can be used on other devices, e.g., valve treatment devices, valve repair devices, etc., that are not necessarily implanted and may be removed after treatment.

In some implementations, there is provided a valve treatment device, which can be an implantable device or implant (e.g., implantable device, etc.), that is configured to be positioned within a native heart valve to help the native heart valve to form a more effective seal. In some implementations, the valve treatment device is part of a valve treatment system including a delivery system having a catheter and a control handle wherein the valve treatment device is coupled to the delivery system.

In some implementations, a valve treatment device (such as an implantable device or implant) includes an anchor portion. Each anchor includes a plurality of paddles that are each moveable between an open position and a closed position.

In some implementations, a valve treatment device (e.g., an implantable device or implant) includes a coaptation element, a first anchor assembly configured to grasp a first leaflet of a native heart valve, and a second anchor assembly configured to grasp a second leaflet of the native heart valve.

In some implementations, the first anchor assembly is connected to a first side of the coaptation element, and the second anchor assembly is connected to a second side of the coaptation element.

In some implementations, the valve treatment device is an implantable device or implant that includes a bioabsorbable central portion between the first anchor assembly and the second anchor assembly. In some implementations, the bioabsorbable central portion includes the coaptation portion.

In some implementations, the bioabsorbable central portion includes a first alignment member associated with the first anchor assembly and a second alignment member associated with the second anchor assembly.

In some implementations, the bioabsorbable central portion includes a coupling element configured to connect the coaptation element, the first anchor assembly, and the second anchor assembly together.

In some implementations, the coupling element threadedly engages at least one of the first alignment member and the second alignment member.

In some implementations, the coupling element extends through a first longitudinal passage in the coaptation element, a second longitudinal passage in the first alignment member, and into a third longitudinal passage in the second alignment member.

In some implementations, the first anchor assembly includes a first inner paddle, and the second anchor assembly includes a second inner paddle. In some implementations, the first alignment member is attached to the first inner paddle and the second alignment member is attached to the second inner paddle.

In some implementations, when the implantable device or implant in an assembled condition, the first alignment member engages the second inner paddle, and the second alignment member engages the first inner paddle.

In some implementations, the first alignment member and the second alignment member are each formed as rectangular blocks.

In some implementations, the coaptation element includes a plurality of lateral passages used to route actuation lines connected to the first and the second anchor assemblies during deployment of the device.

In some implementations, the plurality of lateral passages includes a first pair of lateral passages for routing a first actuation line connecting the first anchor assembly to the coaptation element and a second pair of lateral passages of routing a second actuation line connecting the first anchor assembly to the coaptation element.

In some implementations, the plurality of lateral passages includes a third pair of lateral passages for routing a third actuation line connecting the second anchor assembly to the coaptation element and a fourth pair of lateral passages of routing a fourth actuation line connecting the second anchor assembly to the coaptation element.

In some implementations, the first anchor assembly includes an inner paddle and an outer paddle connected to inner paddle by a bendable portion and the outer paddle is moveable between an open condition and a closed condition relative to the inner paddle.

In some implementations, the first anchor assembly includes a gripping member having a fixed arm attached to the outer paddle and a moveable arm connected to the fixed arm by a joint portion and the moveable arm is moveable between an open condition and a closed condition relative to the fixed arm.

In some implementations, the first anchor assembly is actuatable between an open condition and a closed condition independent of the second anchor assembly.

In some implementations, during deployment of the device, the first anchor assembly is connected to the coaptation element by a plurality of actuation lines.

In some implementations, during deployment of the device, the first anchor assembly can selectively be engaged with the coaptation element and disengaged with the coaptation element while remaining connected to the coaptation element by the plurality of actuation lines.

In some implementations, when the first anchor assembly is disengaged from the coaptation element, the coaptation element and the second anchor assembly are movable relative to the first anchor assembly.

In some implementations, a method of treating and/or repairing a native heart valve includes connecting a first anchor assembly to a coaptation element by a first actuation line, connecting a second anchor assembly to the coaptation element by second actuation line, and positioning a valve treatment device or valve repair device at the native heart valve,

In some implementations, the method includes attaching a first anchor assembly of the valve treatment device/valve repair device to a first valve leaflet of the native heart valve, and attaching a second anchor assembly of the valve treatment device/valve repair device to a second valve leaflet of the native heart valve.

In some implementations, the method includes attaching the first anchor assembly and the second anchor assembly to the coaptation element by a coupling element, and removing the first actuation line and the second actuation line.

In some implementations, positioning the valve treatment device/valve repair device at the native heart valve includes delivering the valve treatment device/valve repair device through a lumen of a catheter where the first anchor assembly, the second anchor assembly, and the coaptation element are axially aligned within the lumen.

In some implementations, the coaptation element and the coupling element are bioabsorbable.

In some implementations, connecting the first anchor assembly to the coaptation element by the first actuation line includes routing the first actuation line through a first pair of lateral passages in the coaptation element. In some implementations, connecting the second anchor assembly to the coaptation element by the second actuation line includes routing the second actuation line through a second pair of lateral passages in the coaptation element.

In some implementations, connecting the first anchor assembly to the coaptation element includes connecting the first anchor assembly to the coaptation element by a third actuation line and routing the third actuation line through a third pair of lateral passages in the coaptation element.

In some implementations, connecting the second anchor assembly to the coaptation element includes connecting the second anchor assembly to the coaptation element by a fourth actuation line and routing the fourth actuation line through a fourth pair of lateral passages in the coaptation element.

In some implementations, the method includes tensioning the first actuation line to draw the first anchor assembly into engagement with the coaptation element. In some implementations, the method includes tensioning the second actuation line to draw the second anchor assembly into engagement with the coaptation element.

In some implementations, attaching the first anchor assembly and the second anchor assembly to the coaptation element by a coupling element includes receiving the coupling element in a first longitudinal passage in the coaptation element, a second longitudinal passage associated with the first anchor assembly, and a third longitudinal passage associated with the second anchor assembly.

In some implementations, the method includes threadedly engaging the coupling element with at least one of the second longitudinal passage and the third longitudinal passage.

In some implementations, attaching the first anchor assembly to the first valve leaflet is completed with the first anchor assembly disengaged from the coaptation element.

In some implementations, attaching the second anchor assembly to the second valve leaflet is completed with the second anchor assembly disengaged from the coaptation element.

In some implementations, attaching the second anchor assembly of the valve repair device to the second valve leaflet includes engaging the coaptation element with the second anchor assembly and moving the coaptation element to move the second anchor assembly in position to attach to the second valve leaflet.

In some implementations, a valve treatment device (such as an implantable device or implant) includes a base, a first anchor assembly connected to a first side of the base, a second anchor assembly connected to a second side of the base, and a connecting member that connects at least the second anchor assembly to the base.

In some implementations, the first anchor assembly is configured to grasp a first leaflet of a native heart valve and the second anchor assembly is configured to grasp a second leaflet of the native heart valve.

In some implementations, the connecting member includes a radiofrequency-reactive material.

In some implementations, a method of treating and/or repairing a native heart valve includes locating a valve treatment device attached to the native heart valve. In some implementations, the method includes disconnecting the second anchor assembly from the base by directing radiofrequency radiation toward the connecting member.

In some implementations, the method includes positioning a replacement valve between the valve leaflets of the native heart valve.

In some implementations, the valve treatment device includes a coaptation member.

In some implementations, the connecting member is a polymer suture embedded with the radiofrequency-reactive material.

In some implementations, the base and the connecting member are formed as a single component.

In some implementations, the base is formed from a polymer material and the radiofrequency-reactive material is embedded in a connection portion of the base that is connected to the second anchor assembly.

In some implementations, the radiofrequency-reactive material is embedded in a center portion of the base.

In some implementations, the radiofrequency-reactive material is an antenna.

In some implementations, a valve treatment device (such as an implantable device or implant) includes a first anchor assembly configured to grasp a first leaflet of a native heart valve and a second anchor assembly configured to grasp a second leaflet of the native heart valve.

In some implementations, the first anchor assembly includes a first connecting member and the second anchor assembly includes a second connecting member. In some implementations, the first connecting member comprises a hook portion of a hook-and-loop fastener and the second connecting member comprises a loop portion of the hook-and-loop fastener.

In some implementations, the first connecting member also includes a loop portion of a hook-and-loop fastener, and the second connecting member also includes a hook portion of the hook-and-loop fastener.

In some implementations, the first and second connecting members include a bioabsorbable material.

In some implementations, a method of treating and/or repairing a native heart valve includes one, some, or all of the following steps of (1) attaching a first anchor assembly to a first leaflet of the native heart valve, (2) attaching a second anchor assembly to a first leaflet of the native heart valve, (3) separating the first and second anchor assemblies with a separation tool, and/or (4) positioning a replacement valve between the valve leaflets of the native heart valve.

In some implementations, the first anchor assembly includes a first connecting member that has a hook portion of a hook-and-loop fastener. In some implementations, the second anchor assembly includes a second connecting member that has a loop portion of a hook-and-loop fastener.

In some implementations, the method further includes a step of compressing the first anchor assembly against the second anchor assembly with an installation tool.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of a valve treatment device, in various stages of deployment;

FIG. 15 shows an example of a valve treatment device that is similar to the device illustrated by FIGS. 8-14, but where the paddles are independently controllable;

FIGS. 16-21 show the example device of FIGS. 8-14 being delivered and deployed within a native valve;

FIG. 22 shows a perspective view of an example device or implant in a closed position;

FIG. 23 shows a perspective view of an example device or implant in a closed position;

FIG. 24 illustrates an example valve treatment device with paddles in an open position;

FIG. 25A illustrates another example valve treatment device with paddles in a closed position;

FIG. 25B illustrates a top view of an example valve treatment device;

FIG. 26 illustrates a perspective view of an example device having paddles of adjustable widths;

FIG. 27 is a cross-section of the example device of FIG. 26 in which the device is bisected;

FIG. 28 is another cross-section of the device of FIG. 26 in which the device is bisected along a plane perpendicular to the plane illustrated in FIG. 28;

FIG. 29 is a schematic illustration of an example implant catheter assembly coupled to a valve treatment device in which an actuation element is coupled to a paddle actuation control and to a driver head of the device;

FIG. 30 is an illustration of the assembly of FIG. 29 with the device rotated 90 degrees to show the paddle width adjustment element coupled to an inner end of the connector of the device and coupled to a paddle width control;

FIG. 31 is an exploded view of an example a valve treatment device or valve repair device.

FIG. 32 is a perspective view of the device of FIG. 31 in an assembled condition;

FIG. 33A is a front perspective view of an example first anchor assembly of the device of FIG. 31;

FIG. 33B is a rear perspective view of the example first anchor assembly of the device of FIG. 31;

FIG. 34A is a front perspective view of an example second anchor assembly of the device of FIG. 31;

FIG. 34B is a rear perspective view of the example second anchor assembly of the device of FIG. 31;

FIG. 35 is a perspective view of an example inner paddle of the device of FIG. 31;

FIG. 36 is a perspective view of an example gripping member of the device of implant of FIG. 31;

FIG. 37 is a perspective view of an example leaflet depth indicator of the device of FIG. 31;

FIG. 38 is a perspective view of an example outer paddle of the device of FIG. 31;

FIG. 39 is a perspective view of an example alignment member of the device of FIG. 31;

FIG. 40 is a perspective view of the first anchor assembly in an open condition;

FIG. 41 is a sectioned perspective view of the coaptation element attached to a coupler;

FIGS. 42-53 are perspective views of the device of FIG. 31 in various states of deployment;

FIG. 54 is a perspective view of an example device with bioabsorbable portions removed;

FIG. 55 shows an example of an implanted treatment device that has been separated into two pieces;

FIG. 56 shows an example of an implanted treatment device that has been separated into two pieces;

FIG. 57 shows an example of an implantable treatment device with anchor assemblies attached to a base with one or more sutures;

FIG. 58 is an enlarged detail view of the sutures of FIG. 57;

FIG. 59 shows the implantable treatment device with one of the anchor assemblies separated from the base;

FIG. 60 shows an example of an implantable treatment device with anchor assemblies attached to a base;

FIG. 61 shows an example of an implantable treatment device with one of the anchor assemblies separated from the base;

FIG. 62 shows a front view of the implantable treatment device of FIG. 60 with a radiofrequency-reactive material embedded throughout the base;

FIG. 63 shows a front view of the implantable treatment device of FIG. 60 with a radiofrequency-reactive material embedded in a portion of the base;

FIG. 64 shows a front view of the implantable treatment device of FIG. 60 with an antenna embedded along the entire length of the base;

FIG. 65 shows a front view of the implantable treatment device of FIG. 60 with an antenna embedded in a portion of the base;

FIG. 66 shows an example of an implantable treatment device with the base separated into two pieces;

FIG. 67 shows a front view of the implantable treatment device of FIG. 66 with a radiofrequency-reactive material embedded in a center portion of the base;

FIG. 68 shows a front view of the implantable treatment device of FIG. 66 with an antenna embedded in the center portion of the base;

FIG. 69 shows a first anchor assembly of an implantable treatment device being implanted onto a first leaflet of a native valve;

FIG. 70 shows a second anchor assembly of an implantable treatment device being implanted onto a second leaflet of the native valve;

FIG. 71 shows the initial connection of the first and second anchor assemblies;

FIG. 72 shows the first and second anchor assemblies fully connected;

FIG. 73 shows the use of an installation tool to facilitate the connection between the first and second anchor assemblies; and

FIG. 74 shows the use of a separation tool to facilitate the separation of between the first and second anchor assemblies.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

Further, the techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

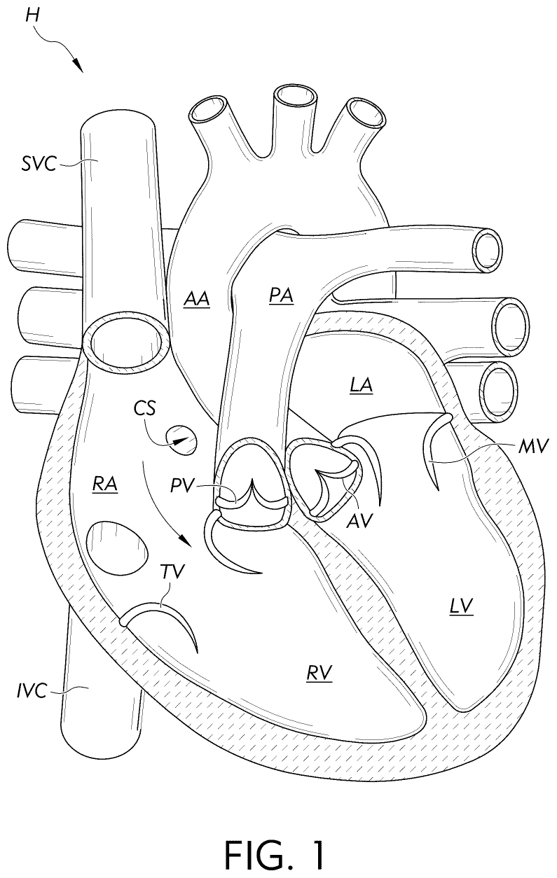

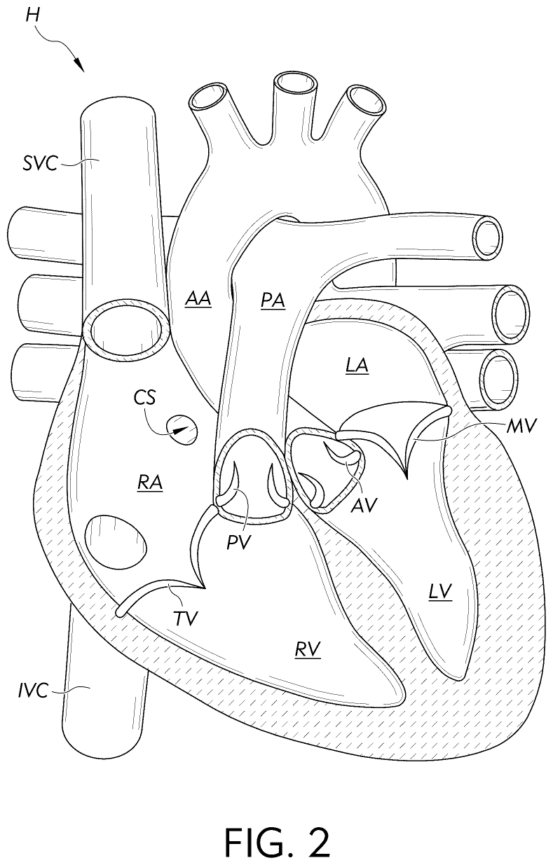

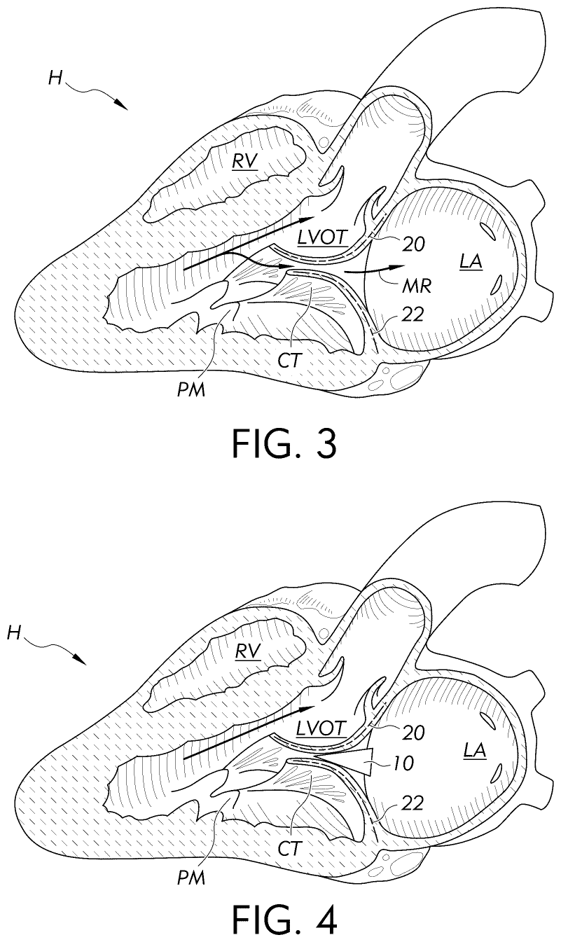

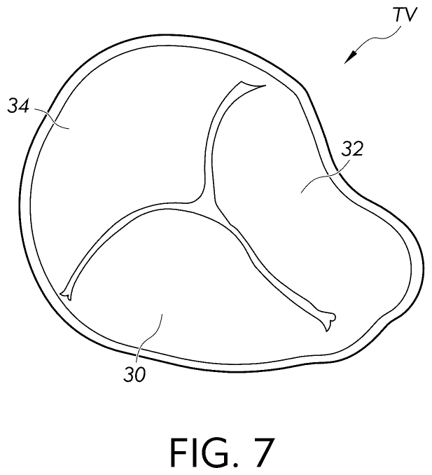

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV, i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

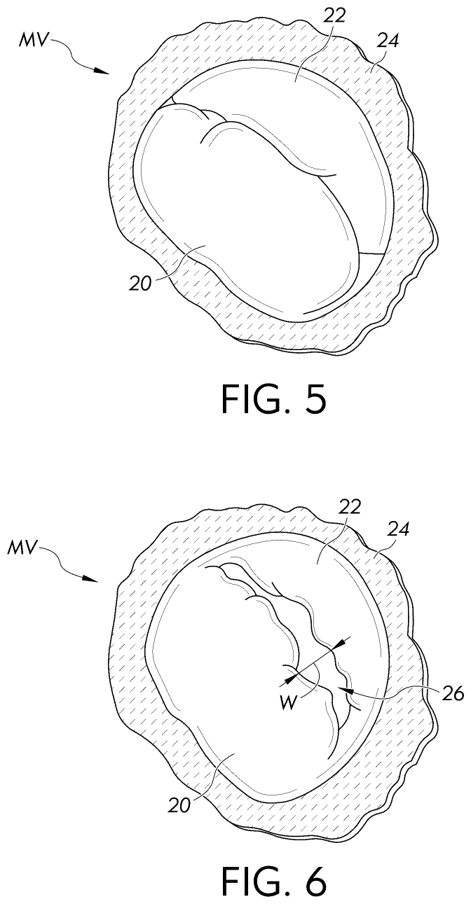

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a valve treatment device or valve repair device (e.g., an implantable device, implant, treatment device, etc.) is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve treatment device/valve repair device, an implantable device, or implant 10 is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve treatment devices or implants provided herein can be centrally located between the three leaflets 30, 32, 34.

An example device or implant can optionally have a coaptation element (e.g., spacer, coaption element, gap filler, membrane, sheet, plug, wedge, balloon, etc.) and at least one anchor (e.g., one, two, three, or more). In some implementations, a valve treatment device/valve repair device (e.g., implantable device, implant, removable treatment device, temporary treatment device, etc.) can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

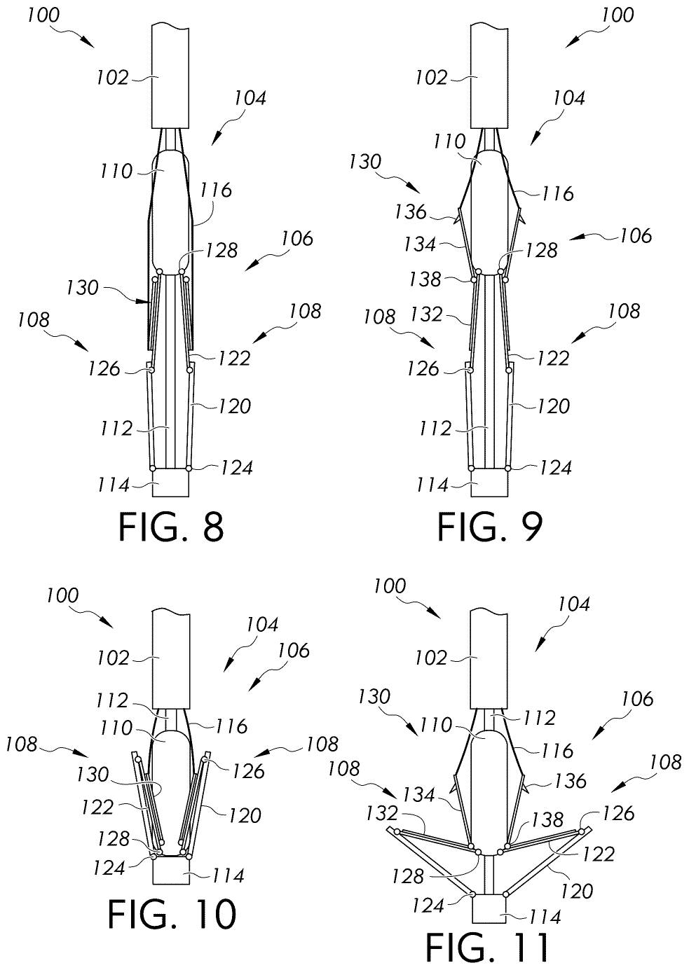

Referring now to FIGS. 8-15, a schematically illustrated device or implant 100 (e.g., a prosthetic device, a valve treatment device, a valve repair device, implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

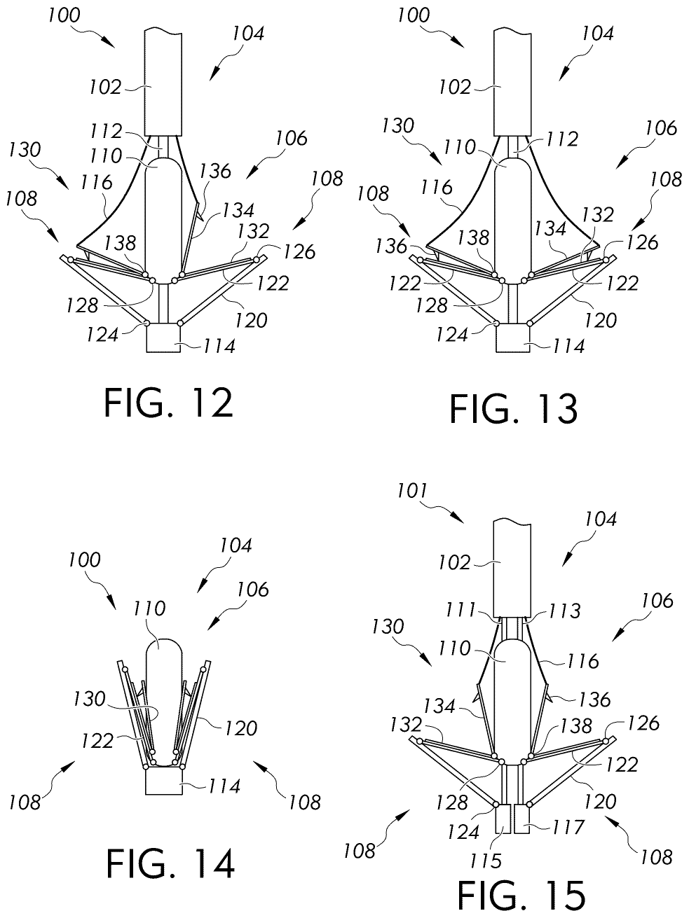

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

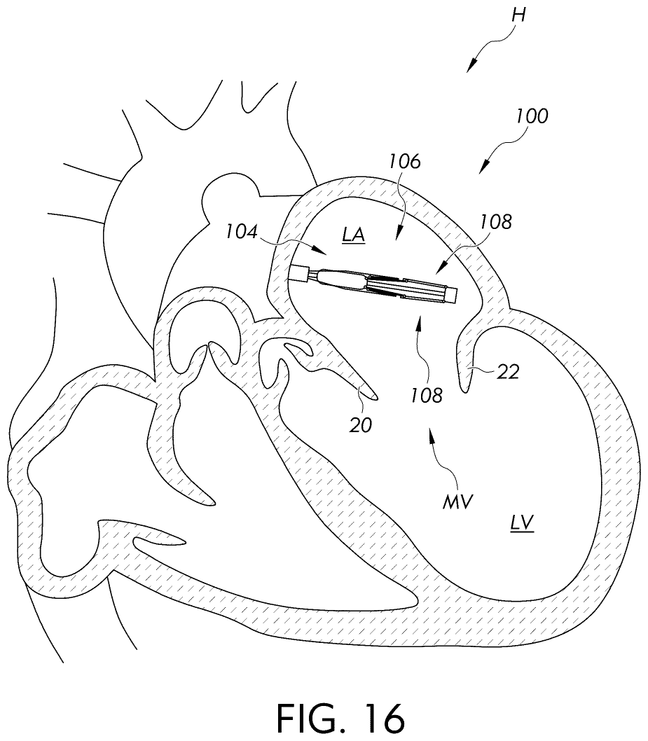

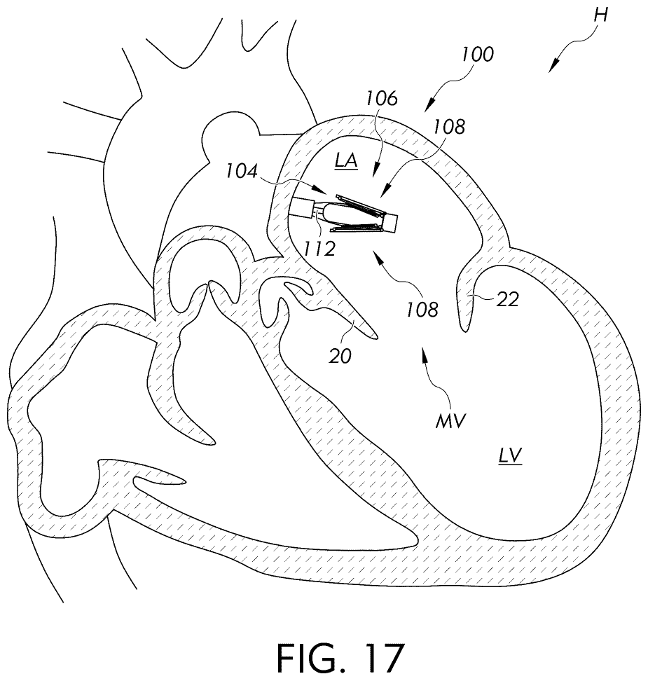

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

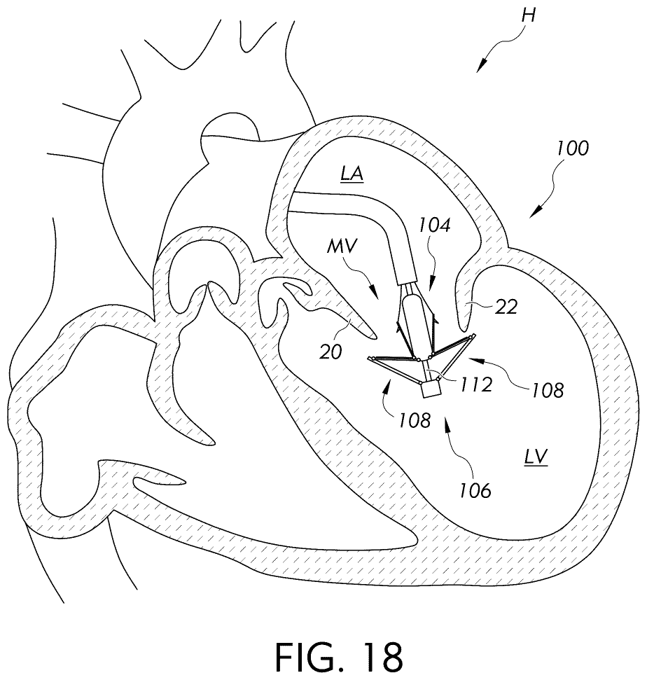

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

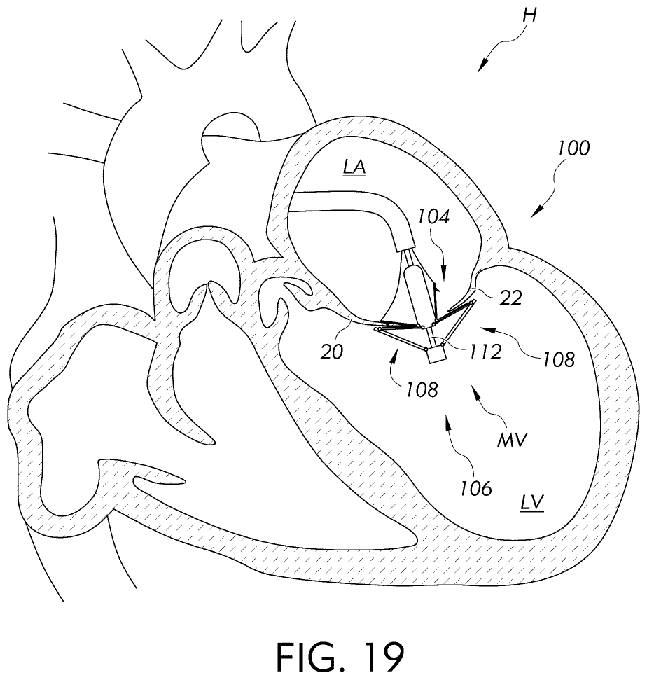

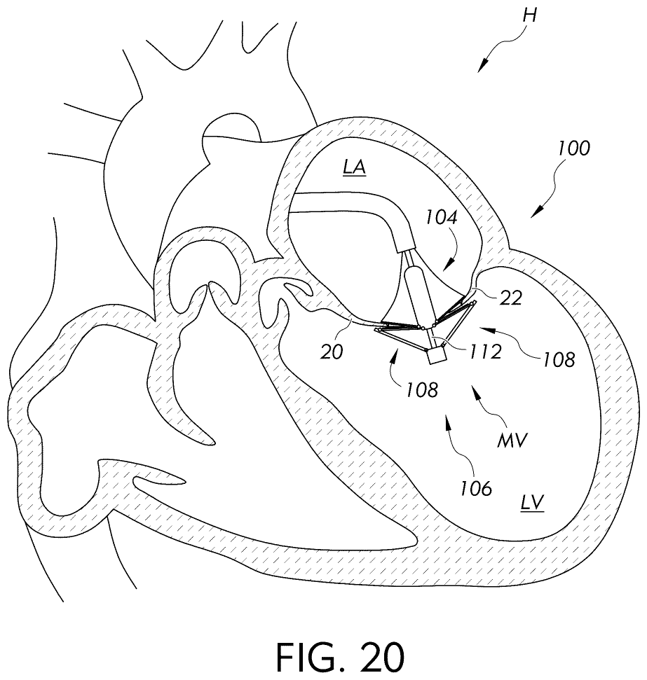

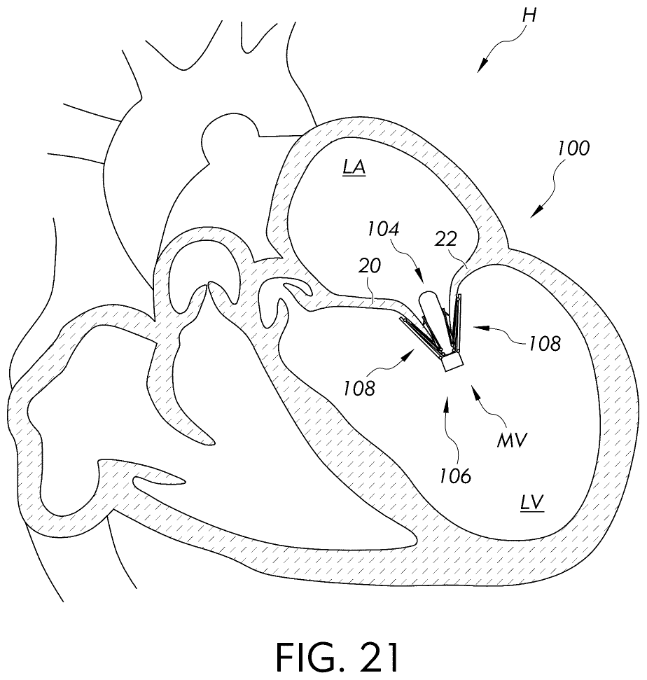

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

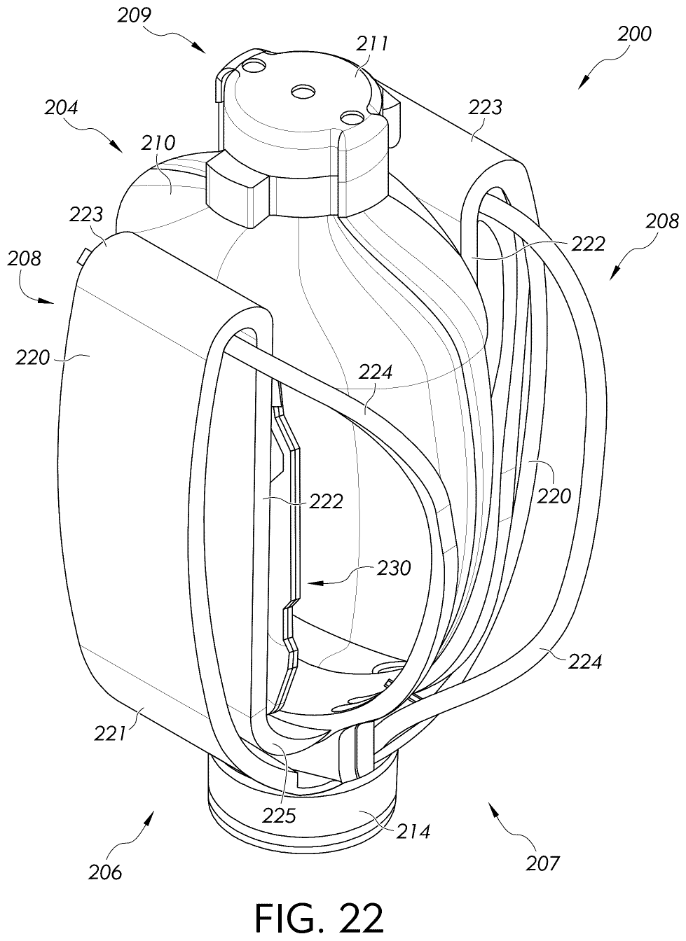

Any of the features disclosed by the present application can be used in a wide variety of different valve treatment devices. FIGS. 22-24 illustrate examples of valve treatment devices that can be modified to include any of the features disclosed by the present application. Any combination or sub-combination of the features disclosed by the present application can be combined with, substituted for, and/or added to any combination or sub-combination of the features of the valve treatment devices illustrated by FIGS. 8-24.

Referring now to FIG. 22, an example of a valve treatment device described for illustrative purposes as an implantable device or implant 200 is shown. The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable valve treatment system or valve repair system (e.g., any valve treatment system or repair system disclosed in the present application). The device/implant 200 can be a prosthetic spacer device, valve treatment device, or another type of implant that attaches to leaflets of a native valve.

In some implementations, the device or implant 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, plug, wedge, balloon, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

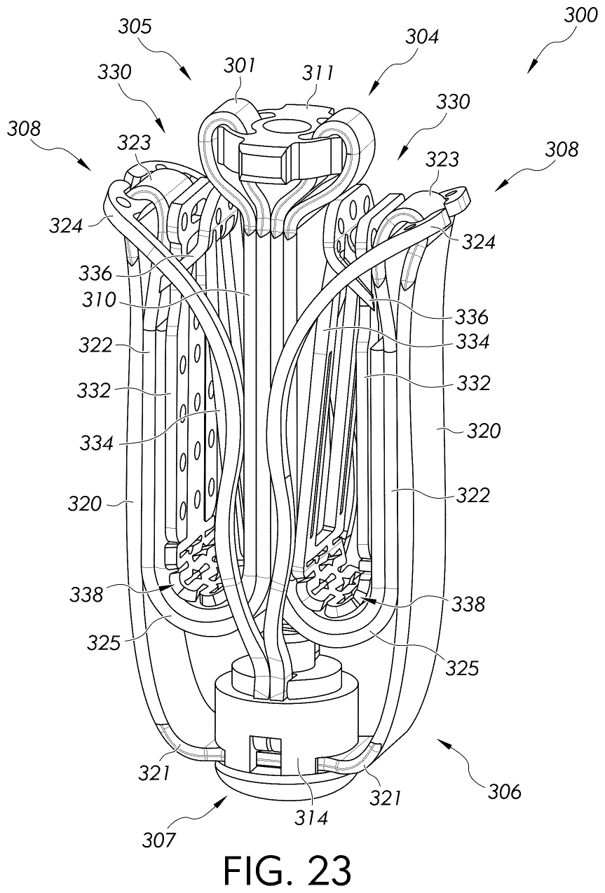

Referring now to FIG. 23, an example of a device or implant 300 is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable valve repair system (e.g., any valve repair system disclosed in the present application).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the device or implant 300 is similar in configuration and operation to the device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

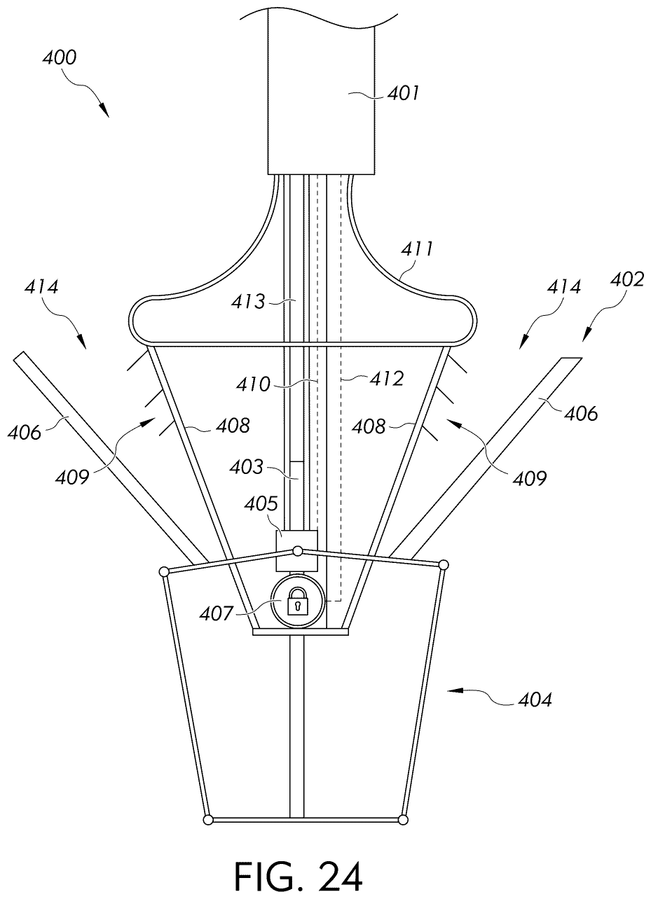

FIG. 24 illustrates another example of one of the many valve treatment systems or valve repair systems 400 for treating or repairing a native valve of a patient that the concepts of the present application can be applied to. The valve treatment system or valve repair system 400 includes a delivery device 401 and a valve treatment device or valve repair device 402.

The valve treatment device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the valve treatment device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include a barbed portion 409 for attaching the gripping members to valve tissue when the valve treatment device 402 is attached to the valve tissue. When the paddles 406 are in the closed position, the paddles engage the gripping members 408, such that, when valve tissue is attached to the barbed portion 409 of the gripping members, the paddles secure the valve treatment device 402 to the valve tissue. In some implementations, the gripping members 408 are configured to engage the paddles 406 such that the barbed portion 409 engages the valve tissue member and the paddles 406 to secure the valve treatment device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the valve treatment device 402 can include any suitable number of paddles and gripping members.

In some implementations, the valve treatment system 400 includes a placement shaft 413 that is removably attached to the shaft 403 of the base assembly 404 of the valve treatment device 402. After the valve treatment device 402 is secured to valve tissue, the placement shaft 413 is removed from the shaft 403 to remove the valve treatment device 402 from the remainder of the valve treatment system 400, such that the valve treatment device 402 can remain attached to the valve tissue, and the delivery device 401 can be removed from a patient's body.

The valve treatment system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The valve treatment device 402 is movable from an open position to a closed position. The base assembly 404 includes links that are moved by the coupler 405. The coupler 405 is movably attached to the shaft 403. In order to move the valve treatment device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the valve treatment device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the valve treatment device 402 in the closed position. After the valve treatment device 402 is maintained in the locked condition by the lock 407, the valve treatment device 402 is removed from the delivery device 401 by disconnecting the shaft 403 from the placement shaft 413. In addition, the valve treatment device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

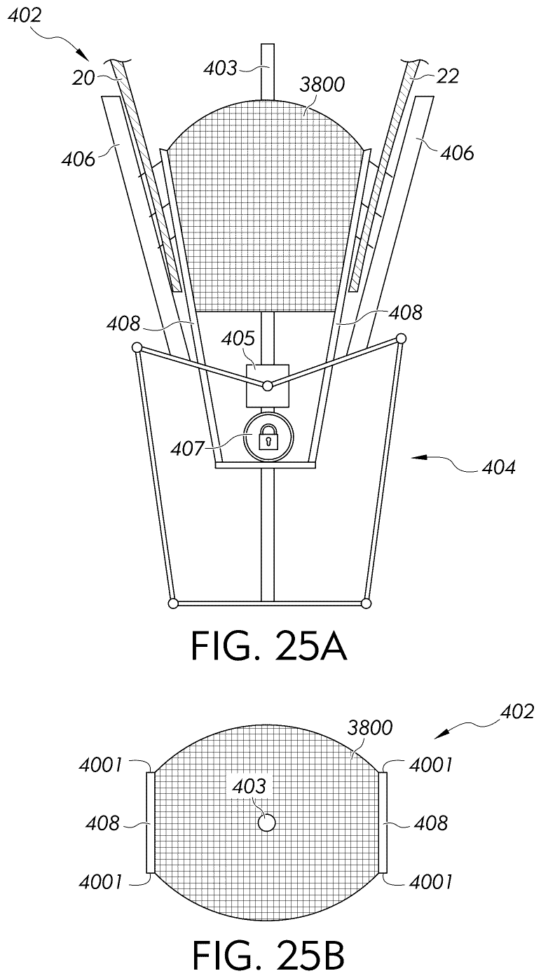

Referring to FIGS. 25A-25B, an example implementation of a valve treatment device 402 has a coaptation element 3800. The valve treatment device 402 can have the same configuration as the valve treatment device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the valve treatment device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the valve treatment device 402 is attached to the leaflets.

The coaptation element 3800 can be disposed around one or more of the shafts or other control elements of the valve treatment system 400. For example, the coaptation element 3800 can be disposed around the shaft 403, the shaft 413, the paddle control mechanism 410, and/or the lock control mechanism 412.

The valve treatment device 402 can include any other features for a valve treatment device or valve repair device discussed in the present application, and the valve repair device 402 can be positioned to engage valve tissue as part of any suitable valve treatment system or valve repair system (e.g., any valve treatment system or valve repair system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

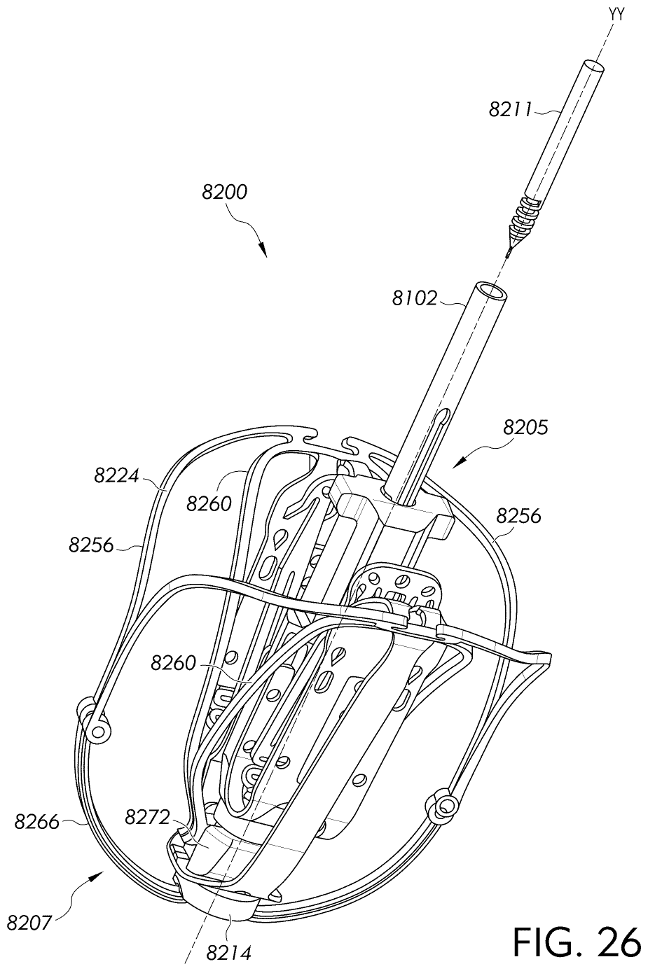

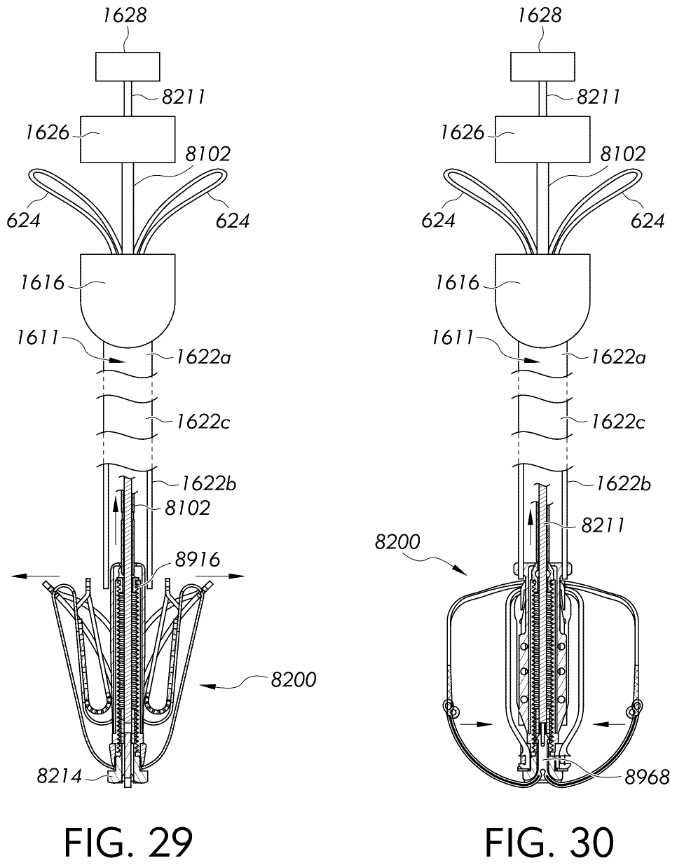

FIGS. 26-30 illustrate an example of one of the many valve treatment systems or valve repair systems for repairing a native valve of a patient that the concepts of the present application can be applied to. Referring to FIGS. 29 and 30, the valve treatment system includes an implant catheter assembly 1611 and an implantable valve treatment device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

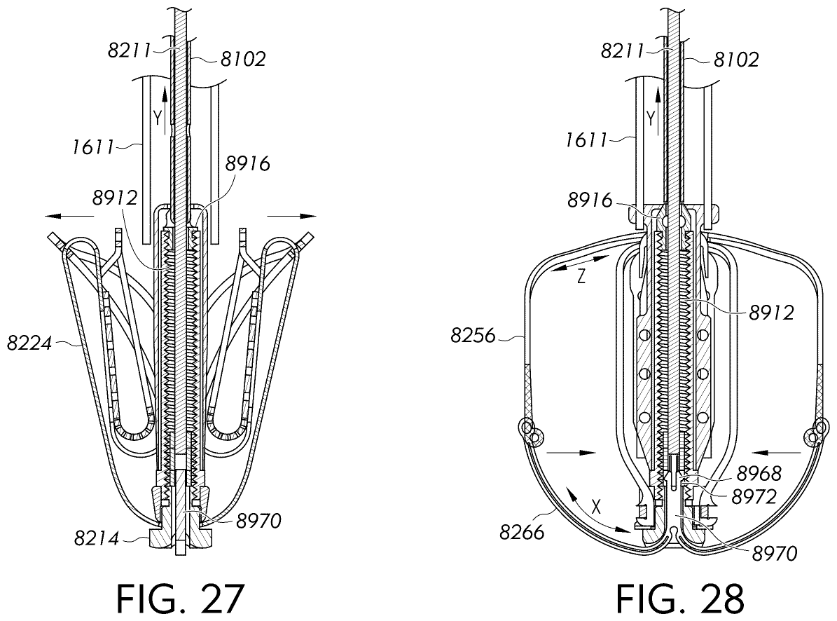

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as the other implementations disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the implant catheter assembly 1611, and through the proximal end of the device 8200, where it couples with the driver head 8916. The actuation element 8102 can be axially movable relative to the outer shaft of the implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

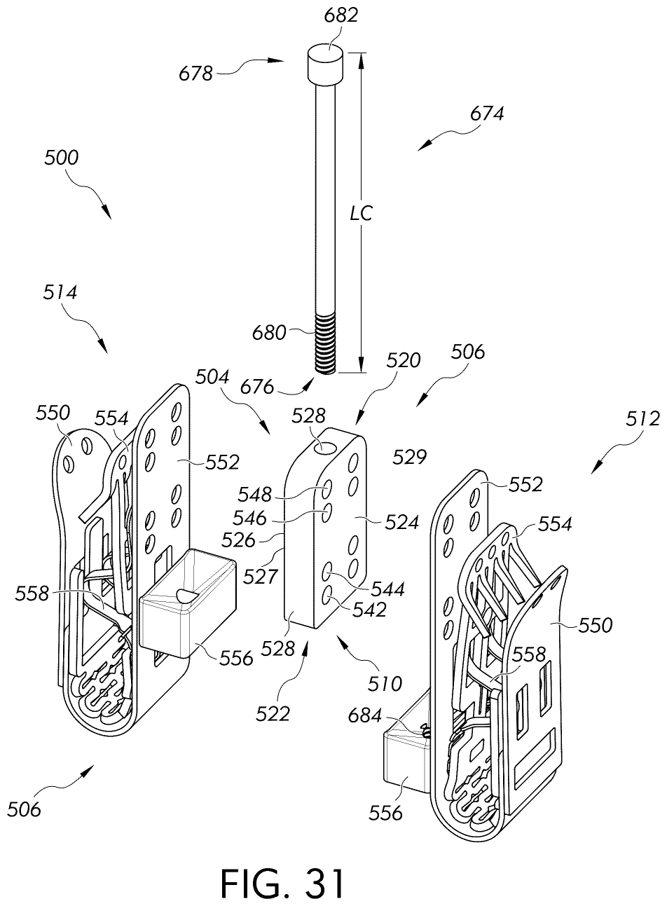

Referring now to FIGS. 31-41, an example device 500 (e.g., a prosthetic device, a valve treatment device, implantable device, implant, etc.) is shown. The device 500 can include any features for another device or implant discussed in the present application or the applications cited above, and the device 500 can be positioned to engage valve tissue (e.g., leaflets 20, 22, 30, 32, 34) as part of any suitable valve treatment system or valve repair system (e.g., any valve treatment system or valve repair system disclosed in the present application, or the applications cited above).

The device or implant 500 is deployed from a delivery system. The delivery system can be any suitable delivery system (e.g., any delivery system disclosed in the present application, or the applications cited above, such as the delivery system 102 of FIGS. 8-20). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 500 includes proximal end 502 and a distal end 503. The device or implant 500 can include an optional coaptation portion 504 and an anchor portion 506. The coaptation portion 504 and the anchor portion 506 can be configured in a variety of ways.

In some implementations, the coaptation portion 504 of the device or implant 500 includes a coaptation element 510 (e.g., spacer, coaption element, gap filler, membrane, sheet, plug, wedge, block, etc.) that is adapted to be implanted between the leaflets of a native valve (e.g., a native mitral valve, native tricuspid valve, etc.). The anchor portion 506 is configured to connect to the coaptation element 510. In some implementations, the anchor portion 506 includes one or more anchor assemblies 512, 514 that are actuatable between open and closed conditions and can take a wide variety of forms, such as, for example, paddles, gripping elements, and/or the like. In the illustrated implementation, the anchor portion 506 includes a first anchor assembly 512 and a second anchor assembly 514.

In some implementations, in an assembled condition, the coaptation element 510 is sandwiched between the first anchor assembly 512 and the second anchor assembly 514 such that the coaptation element 510 defines a center portion 516 of the device or implant 500. In some implementations, all, or a portion, of the coaptation portion 504 is bioabsorbable. Thus, in some implementations, the central portion 516 of the device or implant 500 can be bioabsorbable. Any of the devices or implants discussed in the present application or the applications cited above, can be configured to include a bioabsorbable central portion 516. In some implementations, a portion of the anchor portion 506 can optionally also be bioabsorbable.

In some implementations, the first anchor assembly 512 and the second anchor assembly 514 contact or engage the optional coaptation element 510 in an assembled condition. In some implementations, the first anchor assembly 512 and/or the second anchor assembly 514 can be selectively moved between a first position in which the first anchor assembly 512 and/or the second anchor assembly 514 contacts or engages the coaptation element 510 and a second position in which the first anchor assembly 512 and/or the second anchor assembly 514 is not in contact or is disengaged with the coaptation element 510.

In some implementations, in the second position, each of the first anchor assembly 512 and the second anchor assembly 514 can be positioned in-line with the coaptation element 510 to provide a narrow-delivered configuration when being advanced through the delivery system (i.e., aligned along a longitudinal axis of a lumen of a catheter). In some implementations, in the second position, two or more of the first anchor assembly 512, the second anchor assembly 514, the optional coaptation element 504, and alignment member(s) 556 can be positioned in-line with the coaptation element 510 to provide a narrow-delivered configuration when being advanced through the delivery system (i.e., aligned along a longitudinal axis of a lumen of a catheter).

In some implementations, the first anchor assembly 512 and the second anchor assembly 514 can be independently actuatable. For example, the first anchor assembly 512 can be actuated to grasp a native valve leaflet while the second anchor assembly 514 remains in a ready-to-grasp or open condition. Further, in some implementations, the first anchor assembly 512 can be in the second position (i.e., not in contact with or disengaged from the coaptation element 510) while the second anchor assembly 514 can be in the first position (i.e., in contact or engaged with the coaptation element 510). As a result, the first anchor assembly 512 can be grasping a native valve leaflet in the closed condition and remain in position while the second anchor assembly 514 is moved relative to the first anchor assembly 512, to a position, along with the coaptation element 510, where the second anchor assembly 514 can grasp another native valve leaflet.

Referring to FIG. 31, the coaptation element 510 can be configured in a variety of ways. In the illustrated example, the coaptation element 510 has a generally rectangular body having a proximal end 520, a distal end 522 opposite the proximal end 520, a first face 524 extending between the proximal end 520 and the distal end 522, a second face 526 opposite the first face 524 and extending between the proximal end 520 and the distal end 522, a first side surface 527 extending between the first face 524 and the second face 526, and a second side surface 529 opposite the first side surface 527 and extending between the first face 524 and the second face 526. In some implementations, the first face 524 is parallel to the second face 526.

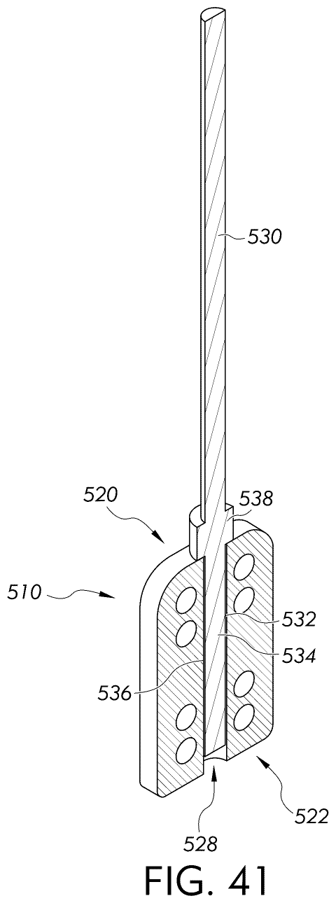

Referring to FIG. 41, in the illustrated example, the coaptation element 510 includes a longitudinal passage 528 extending through the coaptation element 510 from the proximal end 520 to the distal end 522. A coupler 530 (e.g., shaft, wire, tube, hypotube, rod, pusher, etc.) is received within the longitudinal passage 528 to couple to the coaptation element 510. In some implementations, the coupler 530 threadedly engages with the coaptation element 510. For example, in some implementations, the coupler 530 includes male threads 532 on a distal end portion 534 of the coupler 530. The male threads 532 are configured to mate with female threads 536 in the longitudinal passage 528. In some implementations, the coupler 530 can include a stop 538 configured to abut the proximal end 520 to limit how far the coupler 530 can be received within the longitudinal passage 528. The stop 538 can be configured in a variety of ways (e.g., radial shoulder, annular ridge, cross pin or rod, etc.).

Referring to FIGS. 31 and 41, in some implementations, the coaptation element 510 can include a plurality of lateral passages extending through the coaptation element 510 from the first face 524 to the second face 526. The lateral passages can be configured in a variety of ways, including size, number, orientation, and arrangement. In the illustrated implementation, the coaptation element 510 includes a first pair of lateral passages 542, a second pair of lateral passages 544 proximally spaced from the first pair of lateral passages 542, a third pair of lateral passages 546 proximally spaced from the second pair of lateral passages 544, and a fourth pair of lateral passages 548 proximally spaced from the third pair of lateral passages 546. In some implementations, each pair of lateral passages 542, 544, 546, 548 are symmetric about the longitudinal passage 528.

The coaptation element 510 can be made from a bioabsorbable material. The bioabsorbable material can take a variety of different forms. The bioabsorbable material can be a bioabsorbable polymer, such as polyurethane, poly(D,L)lactide, poly(lactic-co-glycolic) acid, poly(α-hydroxy acids), cross-linked polyester hydrogels, poly(orthoesters), polyanhydrides and polyethylene glycol. The bioabsorbable material can be any synthetic material that can be utilized in the valve treatment device/valve repair device 500 that degrades over time in the heart and optionally is replaced with tissue over a period of time due to the device's presence in blood.

The first anchor assembly 512 and the second anchor assembly 514 are each configured to both attach to the coaptation element 510 and to grasp a native valve leaflet. Each of the first anchor assembly 512 and the second anchor assembly 514 can be configured in a variety of ways. The first anchor assembly 512 and the second anchor assembly 514 are actuatable between open and closed conditions and can take a wide variety of forms, such as, for example, paddles, gripping elements, or the like. Referring to FIGS. 31-39, in some implementations, each of the first anchor assembly 512 and the second anchor assembly 514 includes an outer paddle 550, an inner paddle 552, a gripping member 554 (e.g., clasps, clasp arms, grippers, gripping arms, latches, etc.), and an optional leaflet depth indicator 558. In some implementations, an alignment member 556 (e.g., block, body, plug, spacer, etc.) can be associated with each of the first anchor assembly 512 and the second anchor assembly 514.

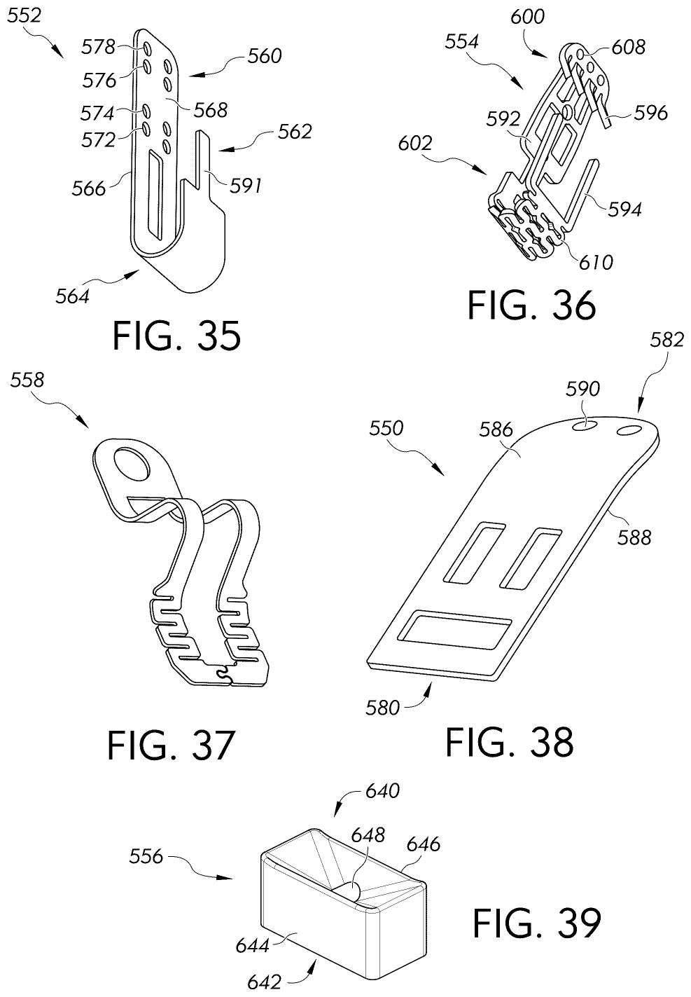

Referring to FIG. 35, the inner paddle 552 can be configured in a variety of ways. In the illustrated example, the inner paddle 552 is configured as a thin, elongated strip having a proximal or first end 560, a distal or second end 562 opposite the proximal end 560, and an intermediate portion 564 between the proximal end 560 and the distal end 562. The inner paddle 552 includes an inner face 566 and an outer face 568 opposite and parallel to the inner face 566. In some implementations, the inner paddle 552 can include a plurality of lateral passages extending through the inner paddle 552 from the inner face 566 and the outer face 568.

The lateral passages can be configured in a variety of ways, including size, number, orientation, and arrangement. In the illustrated implementation, the inner paddle 552 includes a first pair of lateral passages 572, a second pair of lateral passages 574 proximally spaced from the first pair of lateral passages 572, a third pair of lateral passages 576 proximally spaced from the second pair of lateral passages 574, and a fourth pair of lateral passages 578 proximally spaced from the third pair of lateral passages 576.

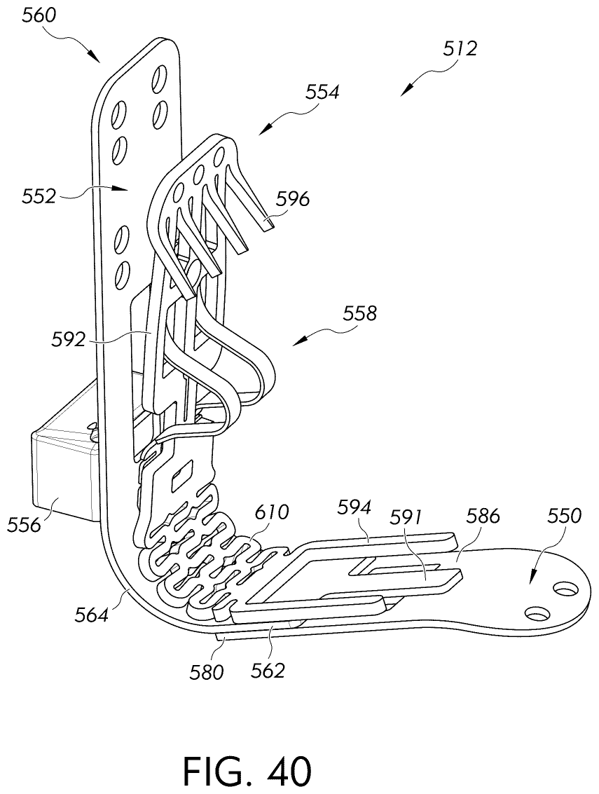

In some implementations, the intermediate portion 564 forms a flexible or bendable region. For example, FIG. 35 illustrates the intermediate portion 564 bent to form a U-shape such that the proximal end 560 is parallel, or generally parallel (e.g., ±10 degrees), to the distal end 562 (i.e., in the closed condition). The intermediate portion 564 can be flexed or bent such that the angle between the proximal end 560 and the distal end 562 is increased. For example, as shown in FIG. 40, the angle between the proximal end 560 and the distal end 562 is a right angle, or generally a right angle (e.g., ±10 degrees) (i.e., in the open condition).

In some implementations, the inner paddle 552 is biased to the closed condition. In some implementations, the inner paddle 552, or a portion thereof (e.g., the intermediate portion 564) can be formed of metals such as steel or shape-memory alloy, such as Nitinol, that are biased to the closed position (e.g., a shape-memory alloy can be shape set to the closed condition).

The outer paddle 550 can be configured in a variety of ways. In the illustrated implementation, the outer paddle 550 is configured as a thin, elongated strip having a first end 580, a second end 582 opposite the first end 580. The outer paddle 550 includes an inner face 586 and an outer face 588 opposite and parallel to the inner face 586. In some implementations, the outer paddle 550 can include a plurality of lateral passages extending through the outer paddle 550 from the inner face 566 and the outer face 568. In the illustrated example, the outer paddle 550 includes a first pair of lateral passages 590 positioned proximate the second end 582.

In some implementations, the inner paddle 552 and the outer paddle 550 can be formed as a single, unitary structure. In other implementations, the outer paddle 550 and the inner paddle 552 can be fixed to each other. For example, as shown in FIG. 40, in the illustrated implementation, the distal end 562 of the inner paddle 552 can be joined to the first end 580 of the outer paddle 550 in a variety of different ways (e.g., directly, indirectly, welding, sutures, adhesive, links, latches, integrally formed, a combination of some or all of these, etc.). In the illustrated implementation, the distal end 562 of the inner paddle 552 includes a narrowed portion 591 configured to be welded to the inner face 586 of the outer paddle.

Referring to FIG. 36, the gripping member 554 can be configured in a variety of ways, including containing features of any of the gripping members disclosed herein. In some implementations, the gripping member 554 can comprise clasps that include a base or moveable arm 592, a fixed arm 594, and optional friction-enhancing elements or other securing structures 596 (e.g., barbs, protrusions, ridges, grooves, textured surfaces, adhesive, etc.).

The moveable arm 592 includes a first end 600 and a second end 602 opposite the first end 600. In some implementations, the moveable arm 592 includes the friction-enhancing elements or other securing structures 596. For example, in the illustrated implementation, the moveable arm 592 includes barbs or other friction-enhancing elements 596 at the first end 600 and facing or projecting radially outward from the first end 600 (e.g., toward the fixed arm 594). In some implementations, the moveable arm 592 can include a plurality of lateral passages 608 extending through the moveable arm 592. In the illustrated implementation, the moveable arm 592 includes three lateral passages 608 positioned at the first end 600.

In some implementations, the moveable arm 592 and the fixed arm 594 are joined by a joint portion 610. The joint portion 610 provides a spring force between the moveable arm 592 and the fixed arm 594. The joint portion 610 can be any suitable joint, such as a flexible joint, a spring joint, a pivot joint, or the like. In some implementations, the joint portion 610 is a flexible piece of material integrally formed with the moveable arm 592 and the fixed arm 594.

The gripping member 554 is actuatable between open and closed conditions. For example, FIG. 36 illustrates the joint portion 610 bent to form a U-shape such that the moveable arm 592 is parallel, or generally parallel (e.g., ±10 degrees), to the fixed arm 594 (i.e., in the closed condition). The joint portion 610 can be flexed or bent such that the angle between the moveable arm 592 and the fixed arm 594 is increased. For example, as shown in FIG. 40, the angle between the moveable arm 592 and the fixed arm 594 is a right angle, or generally a right angle (e.g., ±10 degrees) (i.e., in the open condition).

In some implementations, the gripping member 554 is biased to the closed condition. In some implementations, the gripping member 554, or a portion thereof (e.g., the joint portion 610) can be formed of metals such as steel or shape-memory alloy, such as Nitinol, that are biased to the closed position (e.g., a shape-memory alloy can be shape set to the closed condition).

Referring to FIG. 40, the fixed arm 594 is attached to the outer paddle 550 and remains stationary or substantially stationary relative to the outer paddle 550 when the moveable arm 592 is opened to expose the optional barbs or other friction-enhancing elements 596. The fixed arm 594 can be attached to the outer paddle 550 in a variety of different ways (e.g., directly, indirectly, welding, sutures, adhesive, links, latches, integrally formed, a combination of some or all of these, etc.). In some implementations, the fixed arm 594 can be attached to the distal end 562 of the inner paddle 552 which is attached to the outer paddle 550.

Referring to FIGS. 31-34 and 37, the illustrated implementation includes an optional leaflet depth indicator 558. The optional leaflet depth indicator 558 can be configured in a variety of ways. For example, the leaflet depth indicator 558 can be any of the leaflet depth indicators and can operate in the same or similar manner as any of the leaflet depth indicators disclosed by PCT application no. PCT/US2022/037983, filed on Jul. 22, 2022, which is incorporated herein by reference in its entirety. The illustrated optional leaflet depth indicator 558 is a mechanical leaflet depth indicator that is visually observed (e.g., by fluoroscopy or other known imaging techniques). That is, the arm of the optional mechanical leaflet depth indicator 558 moves based on the presence of the leaflet in the clasp and is observed using an imaging technique to determine the presence or absence of a leaflet in the clasp. In some implementations, the optional leaflet depth indicator 558 can be an electronic leaflet depth indicator that provides a signal that indicates the presence, type, and/or condition of tissue in the clasp. For example, an optional electronic leaflet depth indicator can be any of the electronic leaflet depth indicators disclosed in PCT application no. PCT/US2022/037983. In some implementations, the optional leaflet depth indicator can be a combination of electrical and mechanical/optically observed leaflet depth indicators.

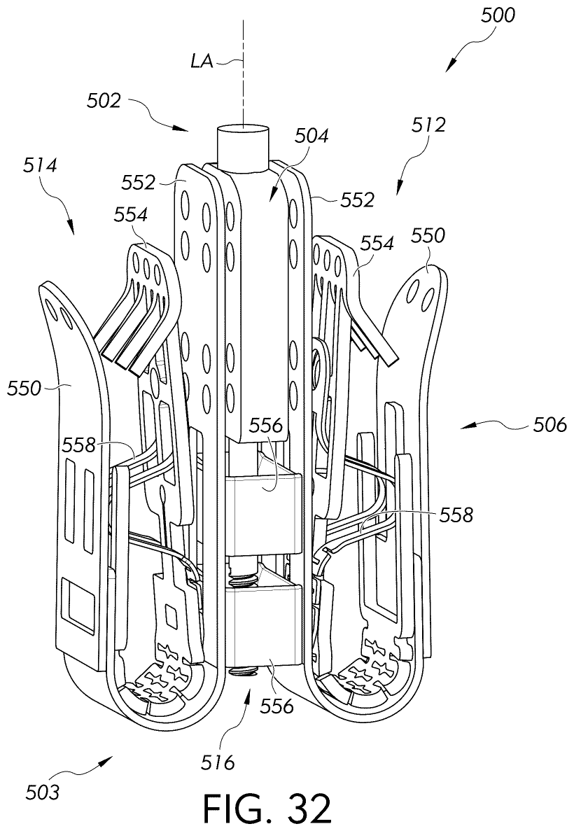

Referring to FIGS. 32, 39-40, the alignment members 556 (e.g., block, body, plug, spacer, etc.) are configured to be positioned between the first anchor assembly 512 and the second anchor assembly 514 when the anchor assemblies 512, 514 are attached to the coaptation element 510. Thus, the alignment members 556, along with the coaptation element 510 at least partially define the central portion 516 of the device 500. The alignment members 556 can be configured in a variety of ways. Referring to FIG. 39, in the illustrated implementation, the alignment member 556 is formed as a rectangular block having top end 640, a bottom end 642 opposite the top end 640, a first face 644 extending between the top end 640 and the bottom end 642, and a second face 646 opposite the first face 644 and extending between the top end 640 and the bottom end 642. In some implementations, a longitudinal passage 648 extends through the alignment member 556 from the top end 640 to the bottom end 642.

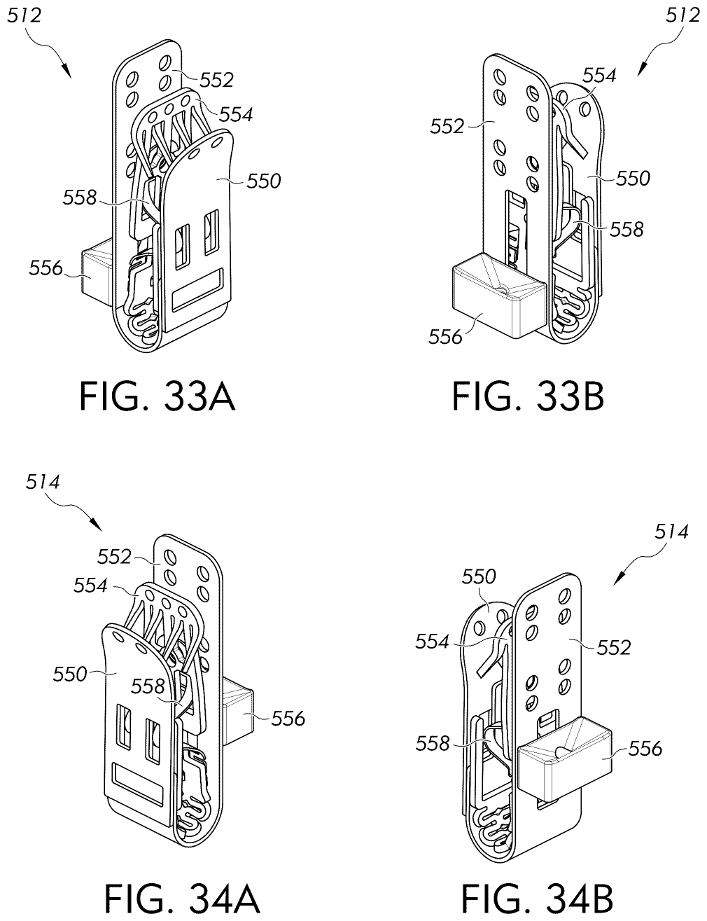

Referring to FIGS. 33A-33B, the first anchor assembly 512 is illustrated in the closed condition. In the illustrated implementation, the second face 646 of the alignment member 556 is attached to the inner face 566 of the inner paddle 552 adjacent, or near, the intermediate portion 564. The alignment member 556 can attach to the inner paddle 552 by any suitable means (e.g., adhesive, fasteners, welding, etc.). The alignment member 556 can be made from a bioabsorbable material. The bioabsorbable material can take a variety of different forms. The bioabsorbable material can be a bioabsorbable polymer, such as polyurethane, poly(D,L)lactide, poly(lactic-co-glycolic) acid, poly(α-hydroxy acids), cross-linked polyester hydrogels, poly(orthoesters), polyanhydrides and polyethylene glycol. The bioabsorbable material can be any synthetic material that can be utilized in the valve treatment device 500 that degrades over time in the heart and optionally is replaced with tissue over a period of time due to the device's presence in blood.

Referring to FIGS. 34A-34B, the second anchor assembly 514 is illustrated in the closed condition. The second anchor assembly 514 is substantially the same as the first anchor assembly 512 so the description of the first anchor assembly 514 applies equally to the second anchor assembly 514. Further, like components of the second anchor assembly 514 use the same reference number as those same components of the first anchor assembly 512.

A difference between the second anchor assembly 514 and the first anchor assembly 512 is the location of the alignment member 556 on the inner face 566 of the inner paddle 552. In particular, the alignment member 556 of the second anchor assembly 514 is positioned more proximal (further from the intermediate portion and close to the that the alignment member of the first anchor assembly 512, as shown in FIGS. 33A-34B. In this way, when in an assembled condition, the alignment members 556 on the first and second anchor assembly 512, 514, stack along the longitudinal axis LA as shown in FIG. 32.

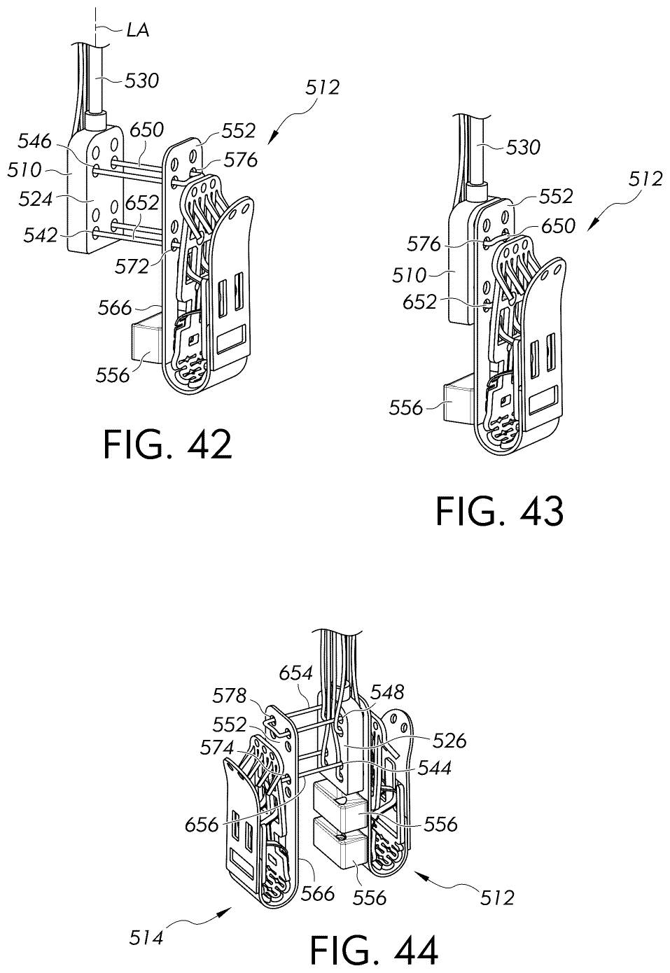

Referring to FIGS. 42-44, the device 500 is illustrated in various stages of deployment and assembly. Referring to FIG. 42, in some implementations, the first anchor assembly 512 is attached to the coaptation element 510 by a plurality of actuation elements (e.g., actuation line, actuation wire, actuation shaft, actuation rod, actuation tube, etc.). In the illustrated implementation, the actuation elements are formed as actuation lines. As shown in FIG. 42, a first actuation line 650 is looped through the third pair of lateral passages 576 on the inner paddle 552 and through the third pair of lateral passages 546 on the coaptation element 510. Similarly, a second actuation line 652 is looped through the first pair of lateral passages 572 on the inner paddle 552 and through the first pair of lateral passages 542 on the coaptation element 510. Applying tension to both ends of the first and second actuation lines 650, 652 pulls the inner face 566 of the inner paddle 552 into engagement with the first face 524 of the coaptation element 510, as shown in FIG. 43. Since the first and the second actuation lines 650, 652 are each looped through aligned pairs of lateral passages, the first anchor assembly 512 is well aligned with the coaptation element 510.

Releasing tension in the first and second actuation lines 650, 652 (i.e., allowing slack) allows the first anchor assembly 512 to disengage from the coaptation element 510, as shown in FIG. 42, while still being connected via the first and second actuation lines 650, 652. Having the first anchor assembly 512 able to disengage from coaptation element 510 can be advantageous. For example, during delivery of the device 500 through the delivery system (e.g., within the lumen of a catheter), the first anchor assembly 512 can be positioned in-line with the coaptation element 510 (e.g., below the coaptation element 510 in FIG. 42 along the longitudinal axis LA). As a result, the device 500 within the lumen of a catheter when being delivered can be smaller than if the first anchor assembly 512 is engaged with the coaptation element 510 as shown in FIG. 43.

Referring to FIG. 44, in some implementations, the second anchor assembly 514 is attached to the opposite side of the coaptation element 510 from the first anchor assembly 512. The second anchor assembly 514 can attach to the coaptation element 510 in a similar way as the first anchor assembly 512 (i.e., by a plurality of actuation elements). In the illustrated implementation, the second anchor assembly 514 is attached to the coaptation element 510 by a plurality of actuation lines (e.g., suture lines). In the illustrated implementation, a third actuation line 654 is looped through the fourth pair of lateral passages 578 on the inner paddle 552 of the second anchor assembly 514 and through the fourth pair of lateral passages 548 on the coaptation element 510. Similarly, a fourth actuation line 656 is looped through the second pair of lateral passages 574 on the inner paddle 552 and through the second pair of lateral passages 544 on the coaptation element 510.

Applying tension to both ends of the third and fourth actuation lines 654, 656 pulls the inner face 566 of the inner paddle 552 into engagement with the second face 526 of the coaptation element 510. Further, the alignment member 556 on the second anchor assembly 514 is positioned between the coaptation element 510 and the alignment member 556 on the first anchor assembly 512 such that the second face 646 the alignment member 556 on the first anchor assembly 512 engages the inner face 566 of the inner paddle 552 of the second anchor assembly and the second face 646 the alignment member 556 on the second anchor assembly 514 engages the inner face 566 of the inner paddle 552 of the first anchor assembly 512. As a result, the alignment members 556 help to ensure the coaptation element 510 and the first and second anchor assemblies are properly aligned with respect to each other when assembled. For ease of illustration, the alignment member 556 on the second anchor assembly 514 is shown detached from the second anchor assembly 514 in FIG. 44.

Releasing tension in the third and fourth actuation lines 654, 656 (i.e., allowing slack) allows the second anchor assembly 514 to disengage from the coaptation element 510, as shown in FIG. 44, while still being connected via the third and fourth actuation lines 654, 656. Thus, similar to the first anchor assembly 512, the second anchor assembly 514 can be positioned in-line with the coaptation element 510 along the longitudinal axis LA for delivery through a lumen of a catheter.

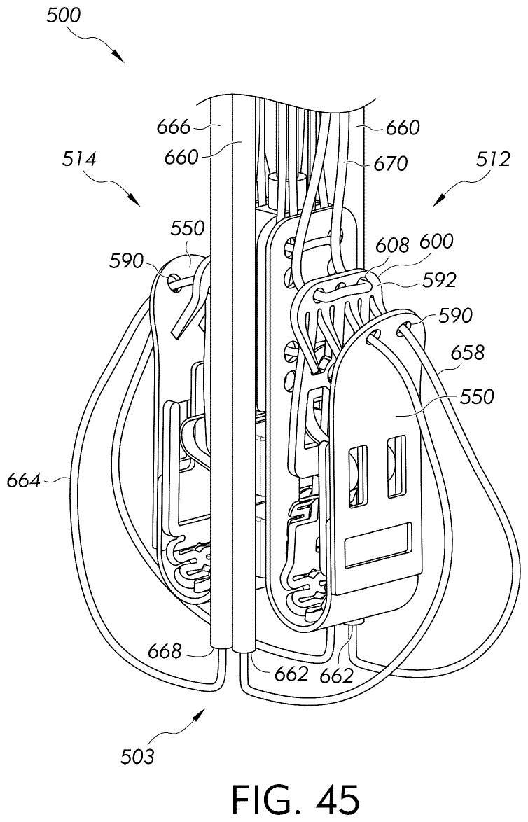

FIGS. 45-49 illustrate the example device 500 being actuated between the open and closed conditions. The device 500 can be actuated by a plurality of actuation elements (e.g., actuation line, actuation wire, actuation shaft, actuation rod, actuation tube, etc.). The actuation elements are omitted in FIGS. 42-44 to simplify these drawings. Referring to FIG. 45, the first anchor assembly 512 and the second anchor assembly 514 are shown attached to the coaptation element 510 as described above in FIGS. 42-44. Each of the first and second anchor assemblies 512, 514, are in the closed condition. The first anchor assembly 512 and the second anchor assembly 514 can be actuated to move between the open and the closed condition in a variety of ways such as any method disclosed herein.

In the illustrated implementation, a fifth actuation line 658 is looped through the first pair of lateral passages 590 on the outer paddle 550 and extends distally to the distal end 503 of the device 500. A first pair of tubes 660 (e.g., hypotubes) extend distally along the device 500 such that an end 662 of each of the first pair of tubes 660 is at, or adjacent the distal end 503. The fifth actuation line 658 extends into the first pair of tubes 660 at the distal end 503. Similarly, a sixth actuation line 664 is looped through the first pair of lateral passages 590 on the outer paddle 550 of the second anchor assembly 514. The sixth actuation line 664 extends distally to the distal end 503 of the device 500. A second pair of tubes 666 (e.g., hypotubes) extend distally along the device 500 such that an end 668 of each of the second pair of tubes 666 is at, or adjacent the distal end 503. The sixth actuation line 664 extends into the second pair of tubes 666 at the distal end 503.

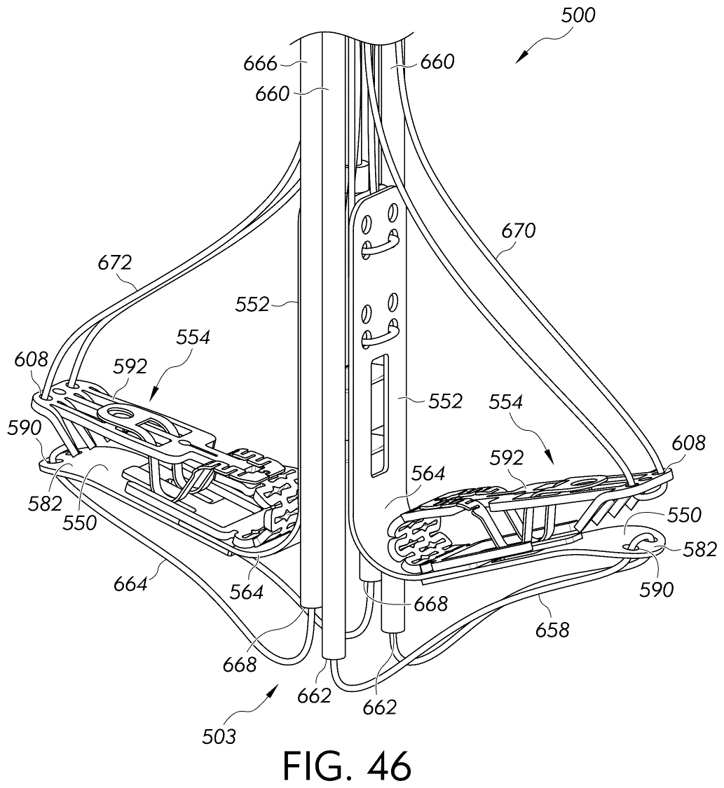

A seventh actuation line 670 is looped through two of the lateral passages 608 at the first end 600 of the moveable arm 592 of the first anchor assembly 512 and extends proximately. Similarly, an eighth actuation line 672 (FIG. 46) is looped through two of the lateral passages 608 at the first end 600 of the moveable arm 592 of the second anchor assembly 514 and extends proximately.

Referring to FIG. 46, to move the outer paddles 550 to an open condition, tension is applied to the fifth actuation line 658 and the sixth actuation line 664 by pulling the lines proximately through the tubes 660, 666, respectively. Since the ends 662, 668 of the tubes 660, 666, respectively, are at or adjacent the distal end 503 of the device 500 and the fifth actuation line 658 and the sixth actuation line 664 are looped through the first pair of lateral passages 590 on the outer paddles 550, tensioning the fifth actuation line 658 and the sixth actuation line 664 provides a distally directed force to the second end 582. As a result, the force causes the intermediate portions 564 to bend or flex to allow the outer paddles 550 to be pivoted away from the inner paddles 552.

As the outer paddles 550 move to the open condition, the gripping members 554 move with the outer paddles 550. To keep the gripping members 554 in the closed condition, the seventh actuation line 670 and the eighth actuation line 672 remain slack to not apply force to the moveable arm 592.

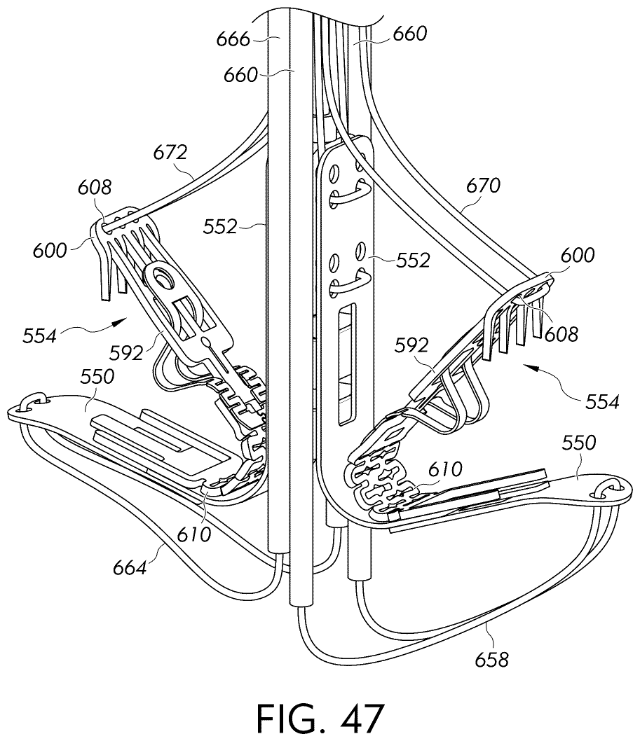

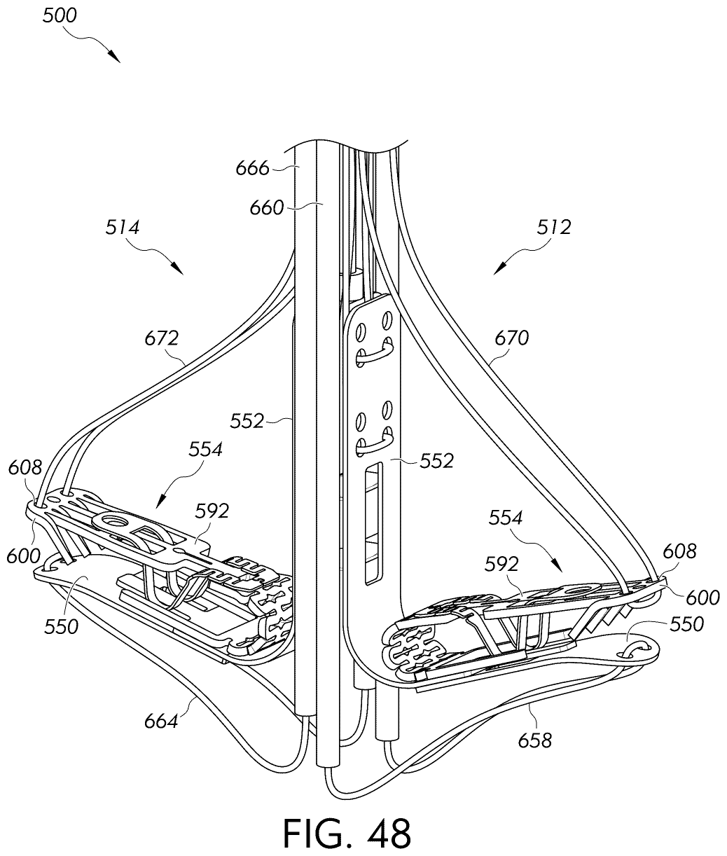

Referring to FIGS. 47-48, to move the gripping members 554 between the closed and open condition, with the outer paddles 550 in the open condition (i.e., tension applied to the fifth actuation line 658 and the sixth actuation line 664), tension can be applied to the seventh actuation line 670 and the eighth actuation line 672. Since the seventh actuation line 670 and the eighth actuation line 672 are looped through the lateral passages 608 at the first end 600 of the moveable arm 592, applying tension to the seventh actuation line 670 and the eighth actuation line 672 provides a proximally directed force to the first end 600 of the moveable arm 592. As a result, the force causes the joint portion 610 to bend or flex to allow the moveable arm 592 to pivot proximately toward the inner paddle 552, as shown in FIG. 47.

In the open condition, each gripping member 554 is capture-ready and can be positioned to capture a valve leaflet. Once positioned to capture a valve leaflet, the tension can be removed from the seventh actuation line 670 and the eighth actuation line 672 allowing the moveable arm 592 to pivot back to the closed condition, as shown in FIG. 48.

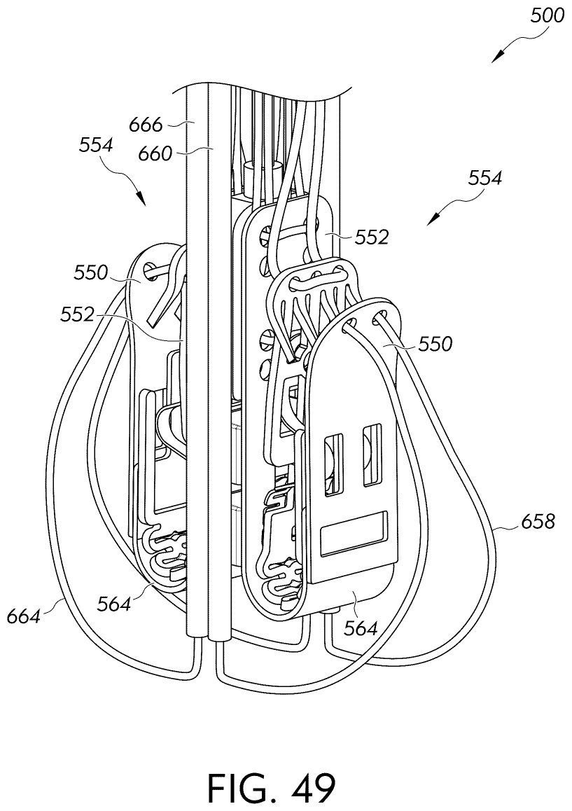

Referring to FIG. 49, with the gripping members 554 in the closed condition, the outer paddles 550 can be moved to the closed condition. In particular, tension can be removed from the fifth actuation line 658 and the sixth actuation line 664 allowing intermediate portion 564 to return the outer paddle 550 to the closed condition.

It will be appreciated that the first anchor assembly 512 and the second anchor assembly 514 are independently actuatable. Thus, the outer and inner paddles 550, 552 and gripping member 554 of the first anchor assembly 512 can be moved between the open and closed conditions independent of the second anchor assembly 514 and vice versa. Further, each of the first anchor assembly 512 and the second anchor assembly 514 can disengage from the coaptation element 510. Thus, for example, the second anchor assembly 514 can capture a first valve leaflet while in a disengaged position from the coaptation element 510, as shown in FIG. 44. The coaptation element 510 and the first anchor assembly 512, which can be engaged with the coaptation element 510, can both be moved via the coupler 530 independent of the second anchor assembly 514 to a position in which the first anchor assembly 512 can capture a second valve leaflet.

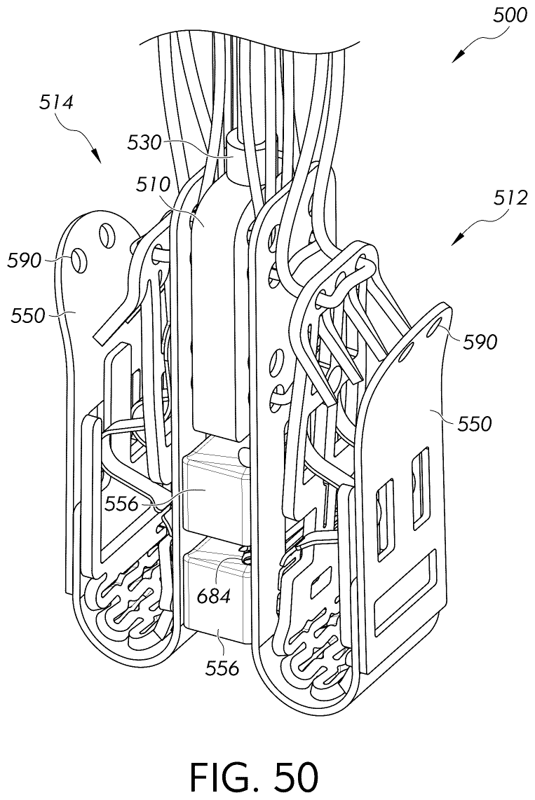

Referring to FIG. 50, after the first and second anchor assemblies 512, 514 are closed, the fifth actuation line 658 and the sixth actuation line 664 can be removed by flossing the fifth actuation line 658 and the sixth actuation line 664 out of the lateral passages 590 in the outer paddles 550 or otherwise removing the actuation lines and withdrawing the tubes 660. The first and second anchor assembly 512, 514 remain attached to the coaptation element 510 via the first, second, third, and fourth actuation lines 650, 652, 654, 656.

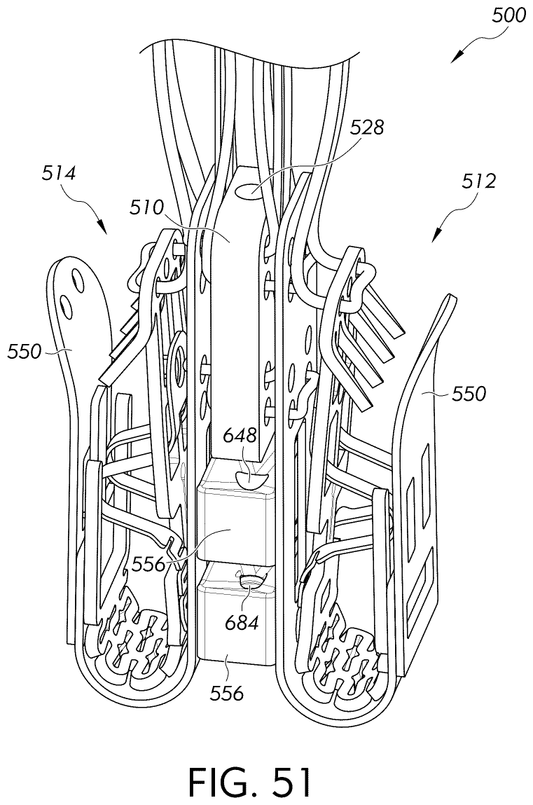

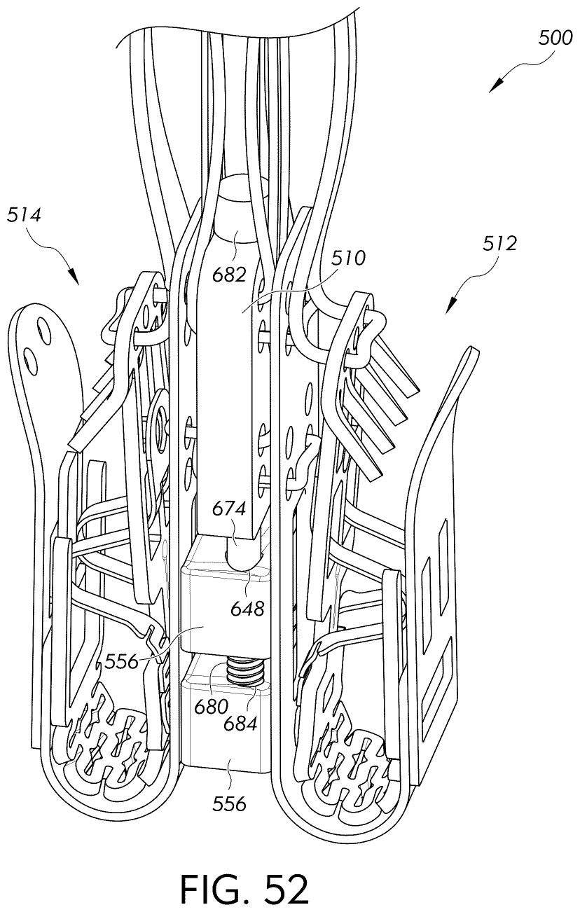

Referring to FIGS. 51-52, prior to removal of the first, second, third, and fourth actuation lines 650, 652, 654, 656, the coupler 530 can be withdrawn from the longitudinal passage 528 (e.g., unthreaded from the female threads 536 in the longitudinal passage 528) and replaced with a coupling element 674 (e.g., pin, wire, shaft, tube, rod, pusher, etc.). As shown in FIG. 51, when the first anchor assembly 512 and the second anchor assembly 514 are attached to the coaptation element 510 the longitudinal passage 528 of the coaptation element 510 aligns with the longitudinal passage 648 in each of the alignment members 556.

The coupling element 674 can be configured in a variety of way. Any configuration that can secure the coaptation element 510, the first anchor assembly 512, and the second anchor assembly 514 together can be used. Referring to FIG. 31, in the illustrated implementation, the coupling element 674 is a cylindrical rod having a distal end 676 and a proximal end 678 opposite the distal end 676. The distal end 676 includes male threads 680 and the proximal end 678 includes an enlarged head 682. The coupling element 674 has a length LC configured to extend from the proximal end 520 of the coaptation element 510, through the coaptation element 510, the alignment member 556 on the second anchor assembly 514, and into the alignment member 556 on the first anchor assembly 512.

In some implementations, the longitudinal passage 648 in the alignment member 556 on the first anchor assembly 512 includes female threads 684 (FIG. 51) configured to mate with the male threads 680 on the coupling element 674. As shown in FIG. 52, the coupling element 674 is received through the longitudinal passage 528 (FIG. 51), through the longitudinal passage 648 in the alignment member 556 on the second anchor assembly 514 and threaded into the longitudinal passage 648 in the alignment member 556 of the first anchor assembly 512 until the head 682 of the coupling element 674 abuts the coaptation element 510.

In some implementations, the coupling element can be omitted and the components of the implanted device 500 can be secured together with one or more of the lines 650, 652, 654, 656. For example, the components of the implanted device 500 can be secured together by applying tension to the lines 650, 652, 654, and/or 656, securing the lines 650, 652, 654, and/or 656 together (e.g., with a lock, anchor, etc. or by tying) in a taught state, and then cutting the lines 650, 652, 654, 656.

In some implementations, the coupling element 674 is made from a bioabsorbable material. The bioabsorbable material can take a variety of different forms. The bioabsorbable material can be a bioabsorbable polymer, such as polyurethane, poly(D,L)lactide, poly(lactic-co-glycolic) acid, poly(α-hydroxy acids), cross-linked polyester hydrogels, poly(orthoesters), polyanhydrides and polyethylene glycol. The bioabsorbable material can be any synthetic material that can be utilized in the valve treatment device 500 that degrades over time in the heart and optionally is replaced with tissue over a period of time due to the device's presence in blood.

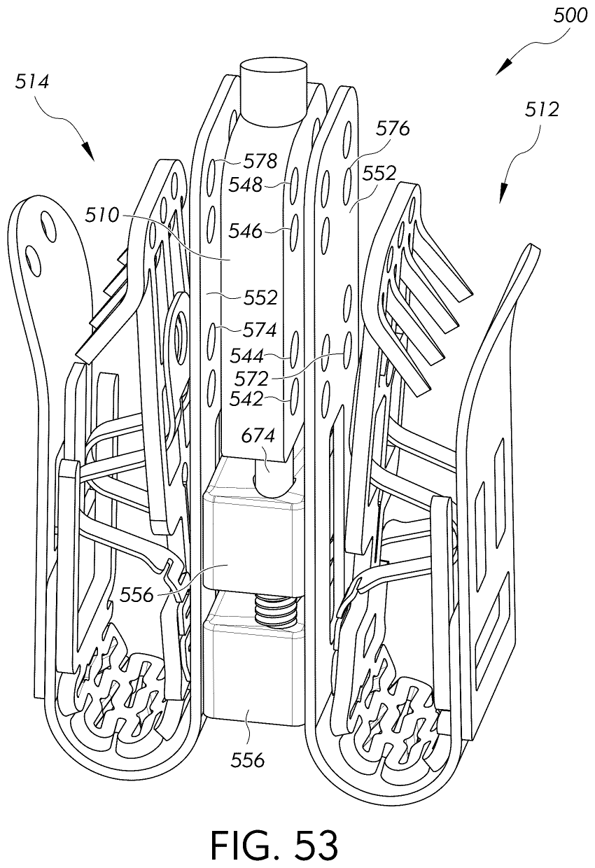

Referring to FIG. 53, once the coupling element 674 is installed and securing the coaptation element 510, the first anchor assembly 512, and the second anchor assembly 514 together, the first, second, third, and fourth actuation lines 650, 652, 654, 656 can be flossed out of the lateral passages 576, 542, 578, 574 in the inner paddles 552, respectively, and the lateral passages 546, 542, 548, 544 in the coaptation element 510, respectively. While FIGS. 49-53 illustrate the tubes 660 and the fifth and the sixth actuation lines 658, 664 being removed prior to the coupler 530 being replaced by the coupling element 674, it should be appreciated that the coupler 530 can be replaced by the coupling element 674 prior to removal of the tubes 660 and the fifth and the sixth actuation lines 658, 664. With the coupling element 674 in place to secure the coaptation element 510, the first anchor assembly 512, and the second anchor assembly 514 together and the first, second, third, and fourth actuation lines 650, 652, 654, 656 removed, the device 500 is in an assembled condition.

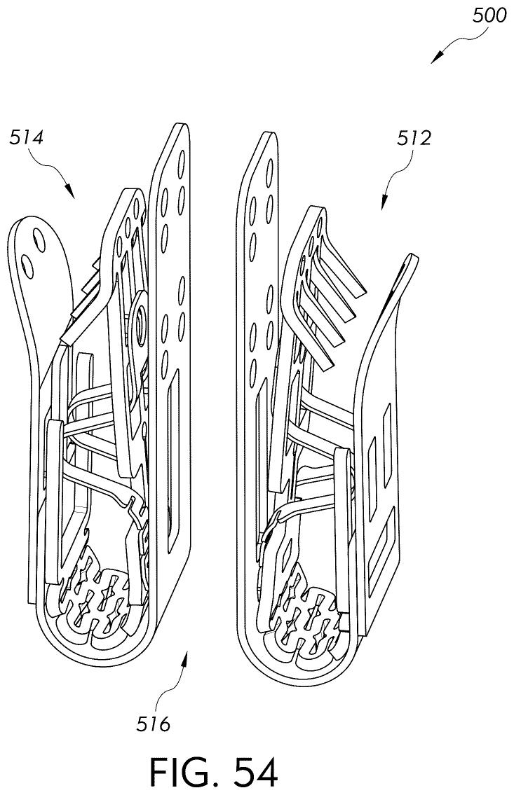

Referring to FIG. 54, the device 500 is illustrated with the bioabsorbable portions (i.e., the coaptation element 510, the alignment members 556, and the coupling element 674), which define the central portion 516 of the device 500, removed. Over time, the bioabsorbable portions of an implanted device 500 will decompose and are replaced by tissue ingrowth. Remaining portions of the device after the bioabsorbable portions decompose are the first and second anchor assemblies 512, 514. An advantage of the device 500 having the central portion 516 defined by bioabsorbable portions is that should the device 500 need to be removed in the future, the first and the second anchor assemblies 512, 514 can be split by cutting through the ingrown tissue that now defines the central portion 516 of the device since the tissue has replaced the bioabsorbable portions of an implanted device 500.

An implanted valve treatment/repair device, such as example devices described herein, that is secured to the leaflets of the native heart valve via anchor assemblies may require removal and/or splitting to facilitate follow-up therapy, such as, for example, a transcatheter valve replacement. That is, a transcatheter replacement valve may not be able to be installed into the native valve when the leaflets are connected by an existing device. To enable the leaflets to be spread apart during installation of the replacement valve, devices such as implantable treatment and/or repair devices herein can be configured to be separated or separable so that anchor assemblies attached to each of the valve leaflets are no longer joined together and/or the leaflets and anchor assemblies can be spread apart during installation of the replacement valve.

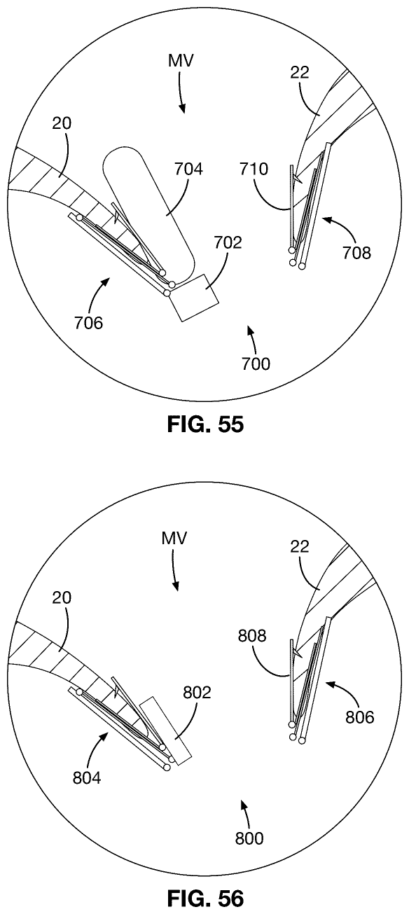

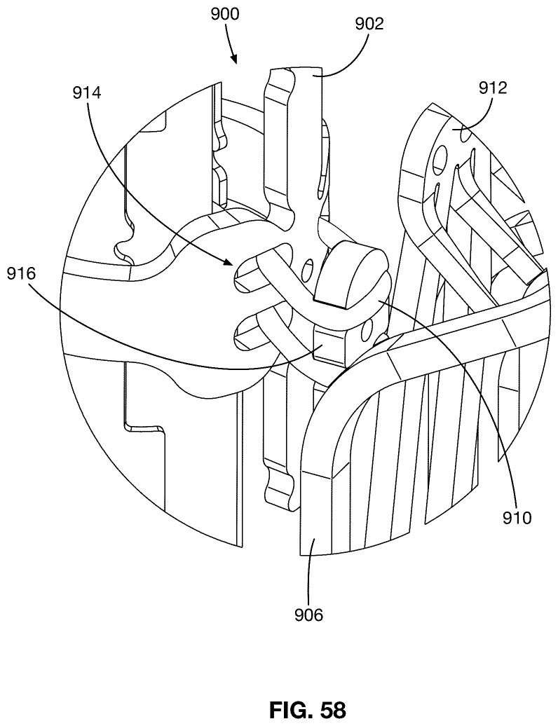

Referring now to FIGS. 55-74, various valve treatment and/or repair devices are shown that can be separated if desired, e.g., after implantation, etc. Any of the implantable devices described herein can incorporate the features of the devices shown in FIGS. 55-74, i.e., these can be added to earlier devices described previously.

Referring now to FIG. 55, an implanted device 700 is shown in a separated condition to enable the implantation of another device or a replacement valve. The device 700 can be the same as or similar to the device 100 described above. In some implementations, device 700 includes a base 702, a coaptation portion 704, a first anchor assembly 706, and a second anchor assembly 708.

In some implementations, each anchor assembly 706, 708 includes a gripping member 710 for securing the anchor assembly 706, 708 to one of the valve leaflets 20, 22. As can be seen in FIG. 55, the second anchor assembly 708 has been separated from the base 702, which can be accomplished by any of the techniques described herein. Separating the second anchor assembly 708 from the base 702 allows the second anchor assembly 708 to move freely with the leaflet 22 to which the second anchor assembly 708 is attached. In some implementations, the remaining components of the device 700—i.e., the base 702, the coaptation portion 704, and the first anchor portion 706—move freely with the other leaflet 20. In other words, separating the second anchor assembly 708 from the base 702 allows the leaflets 20, 22 to move in their untreated condition. Consequently, the leaflets 20, 22 can be captured by a different implantable valve treatment device or can be spread apart by the implantation of a replacement valve.

Referring now to FIG. 56, an example device 800 that was previously implanted is shown in a separated condition to enable the implantation of another device or a replacement valve. The device 800 can be the same as or similar to the devices 100, 700 described above. In some implementations, the device 800 includes a base 802, a first anchor assembly 804, and a second anchor assembly 806. In some implementations and as shown here, the device 800 does not include a coaptation element like the coaptation element 704 of the device 700 shown in FIG. 55.

In some implementations, each anchor assembly 804, 806 includes a gripping member 808 for securing the anchor assembly 804, 806 to one of the valve leaflets 20, 22. As can be seen in FIG. 56, the second anchor assembly 806 has been separated from the base 802, which can be accomplished by any of techniques described herein. Separating the second anchor assembly 806 from the base 802 allows the second anchor assembly 806 to move freely with the leaflet 22 to which the second anchor assembly 806 is attached. The remaining components of the device 800—i.e., the base 802 and the first anchor portion 804—move freely with the other leaflet 20. In other words, separating the second anchor assembly 806 from the base 802 allows the leaflets 20, 22 to move in their untreated condition. Consequently, the leaflets 20, 22 can be captured by a different implantable valve treatment device or can be spread apart by the implantation of a replacement valve.

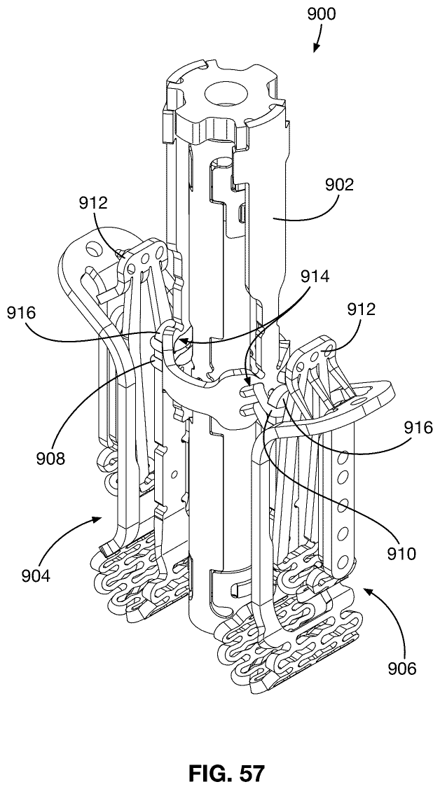

Referring now to FIGS. 57-59, an implantable valve treatment device 900 is shown. The device 900 can include any of the features of any other valve treatment devices described herein. In some implementations, the device 900 includes a base 902 that optionally includes a spacer or a coaptation element, not shown. The optional spacer can be any suitable spacer, such as the spacers and coaptation elements described herein.

In some implementations, the device 900 includes a first anchor assembly 904 and a second anchor assembly 906. In some implementations, the first anchor assembly 904 is attached to the base 902 with a first connecting member 908 and the second anchor assembly 906 is attached to the base 902 with a second connecting member 910. In some implementations, each of the first and second anchor assemblies 904, 906 includes a gripping member 912 for securing the anchor assembly 904, 906 to one of the valve leaflets 20, 22.

In some implementations, the first connecting member 908 and the second connecting member 910 can be formed from any suitable material or component for attaching the anchor assemblies 904, 906 to the base. For example, as can be seen in FIGS. 57-59, the first and second connecting members 908, 910 can take the form of sutures that are routed through one or more attachment openings 914 of the base 902 and around an attachment portion 916 of the anchor assemblies 904, 906.

In some implementations, to facilitate the disconnection of one of the anchor assemblies 904, 906 from the base 902, one of the connecting members 908, 910 can be formed from a polymer material that is embedded with a radiofrequency-reactive material, such as, for example, iron oxide. In some implementations, upon exposure to a radiofrequency field, the radiofrequency-reactive material increases in temperature and causes the polymer material of the connecting member 908, 910 to melt, thereby disconnecting the attached anchor assembly 904, 906 from the base 902.

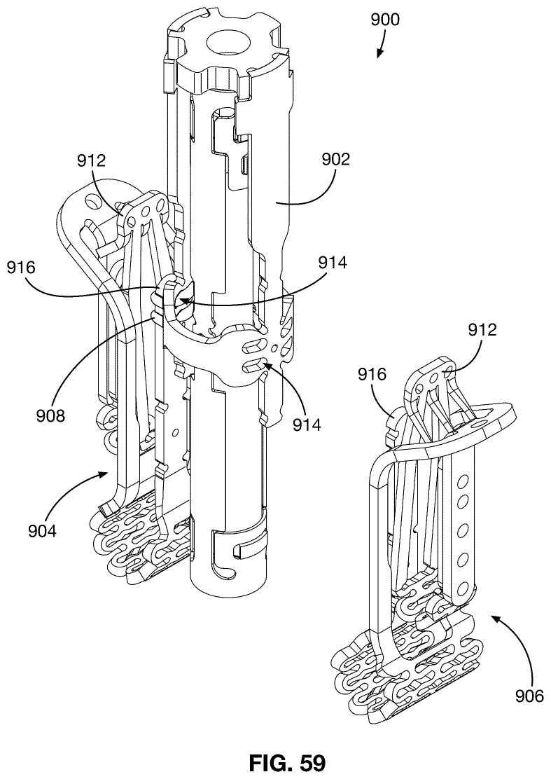

In the illustrated example shown in FIGS. 57-59, the first connecting member 908 is free from a radiofrequency-reactive material and/or is formed integrally with the base 902 and the first anchor assembly 904. In some implementations, the second connecting member 910 is formed from a polymer suture embedded with radiofrequency-reactive material. As is shown in FIG. 59, once the second connecting member 910 has been exposed to a radiofrequency field such that the polymer suture melts, the second anchor assembly 906 is disconnected or detached from the base 902 while the first anchor assembly 904 remains connected and attached to the base 902. In this way, elements of the device 900 are prohibited from detaching from the valve leaflets 20, 22 that are gripped by the gripping members 912 even though the device 900 itself is separated into two pieces.

Following the detachment of the second anchor assembly 906 from the base 902, a new implantable valve repair device or an implantable valve replacement can be installed between the valve leaflets.

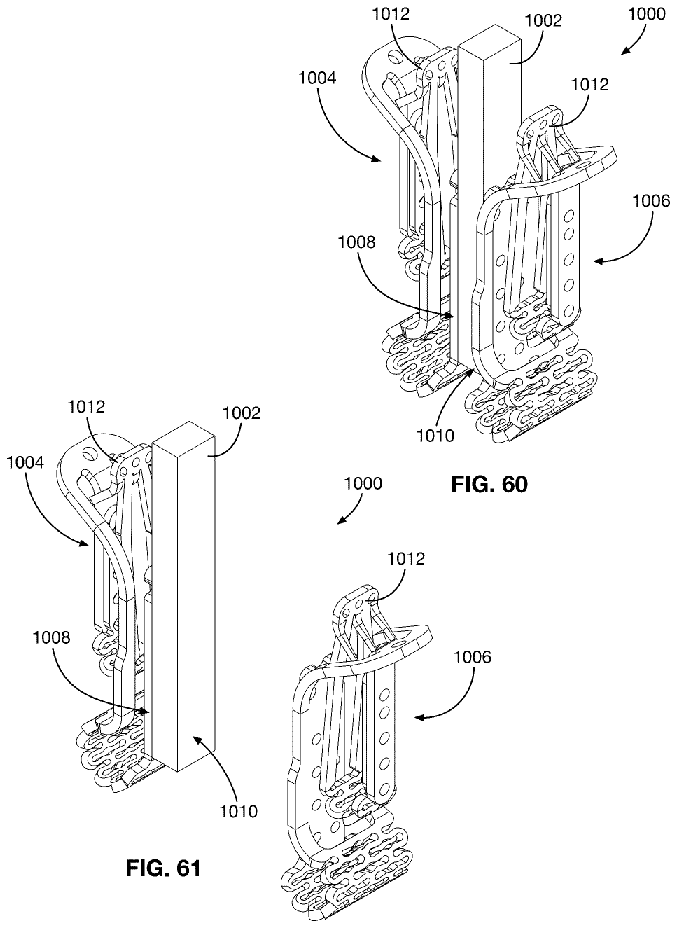

Referring now to FIGS. 60-68, an implantable valve treatment device 1000 is shown. The device 1000 can include any of the features of any other valve treatment devices described herein. In some implementations, the device 1000 includes a base 1002 that optionally includes a spacer or a coaptation element, not shown. The optional spacer can be any suitable spacer, such as the spacers and coaptation elements described herein.

In some implementations, the device 1000 includes a first anchor assembly 1004 and a second anchor assembly 1006. In some implementations, the first anchor assembly 1004 is attached to the base 1002 at a first attachment location 1008 and the second anchor assembly 1006 is attached to the base 1002 at a second attachment location 1010.

In some implementations, the first and second anchor assemblies 1006, 1008 can be attached to the first and second attachment locations 1008, 1010 of the base 1002 in any suitable way. For example, the first and second anchor assemblies 1006, 1008 can be integrally formed with the base 1002, or can be secured to the base 1002 via an adhesive, or can be embedded in the base 1002. Each of the first and second anchor assemblies 1004, 1006 includes a gripping member 1012 for securing the anchor assembly 1004, 1006 to one of the valve leaflets 20, 22.

Referring now to FIG. 61, the second anchor assembly 1006 is shown separated from the base 1002 similar to the devices 800 and 900 shown in FIGS. 55-59. The base 1002 can be formed from any suitable material, such as, for example, a polymer material that is partially embedded with a radiofrequency-reactive material 1020 similar to the second connecting member 910 described herein.

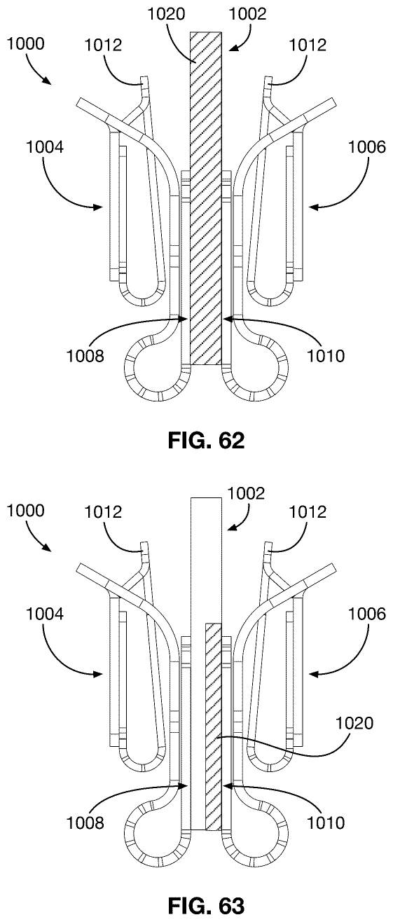

Referring now to FIGS. 62-63, different arrangements of the radiofrequency-reactive material 1020 in the base 1002 are shown. Each of the arrangements of the base 1002 shown in FIGS. 62-63 is configured to separate one or both of the first anchor assembly 1004 and the second anchor assembly 1006 from the base 1002.

In some implementations, the base 1002 in FIG. 62 includes a radiofrequency-reactive material 1020 embedded throughout the polymer base 1002 so that exposure to a radiofrequency field increases the temperature of the entire base 1002 so that the material of the base 1002 and both of the first and second attachment locations 1008, 1010 melts, thereby disconnecting both the first anchor assembly 1004 and the second anchor assembly 1006. In the example of FIG. 63, the radiofrequency-reactive material 1020 is embedded in the second attachment location 1010 so that exposure to a radiofrequency field increases the temperature of the second attachment location 1010 so that the material of the second attachment location 1010 melts to separate the second anchor assembly 1006 from the base 1002.

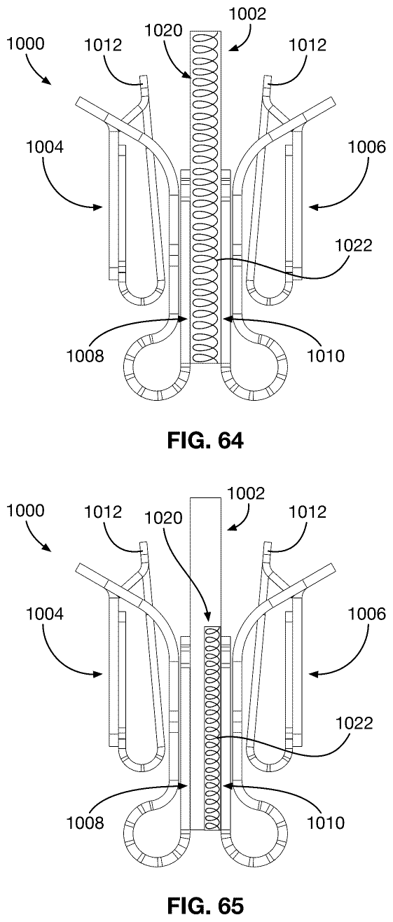

Referring now to FIGS. 64-65, the radiofrequency-reactive material 1020 embedded in the base 1002 is shown as an antenna 1022 that increases in temperature when exposed to a radiofrequency field. The antenna 1022 can be tuned to respond to a particular radiofrequency or a range of radiofrequencies. Each of the arrangements of the base 1002 shown in FIGS. 64-65 is configured to separate one or both of the first anchor assembly 1004 and the second anchor assembly 1006 from the base 1002.

In some implementations, the base 1002 in FIG. 64 includes an antenna 1022 embedded throughout the polymer base 1002 so that exposure to a radiofrequency field increases the temperature of the entire base 1002 so that the material of the base 1002 and both of the first and second attachment locations 1008, 1010 melts, thereby disconnecting both the first anchor assembly 1004 and the second anchor assembly 1006. In FIG. 65, the antenna 1022 is embedded in the second attachment location 1010 so that exposure to a radiofrequency field increases the temperature of the second attachment location 1010 so that the material of the second attachment location 1010 melts to separate the second anchor assembly 1006 from the base 1002.

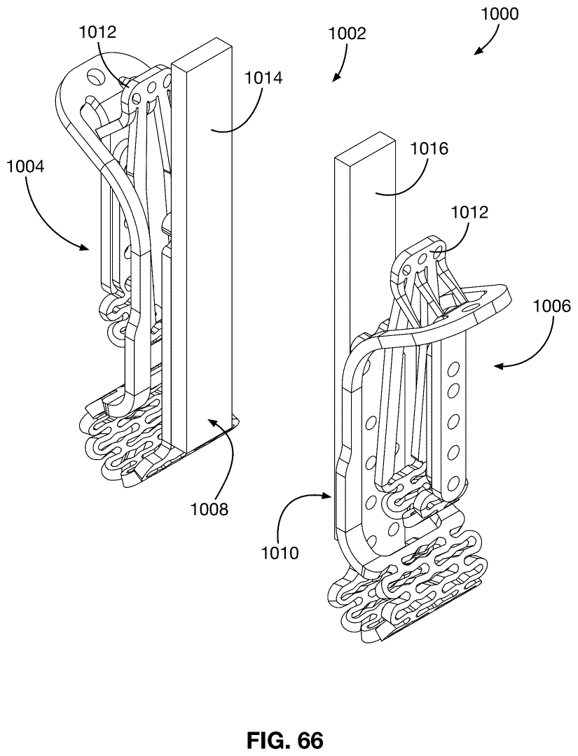

Referring now to FIGS. 66-68, the base 1002 is shown split into two pieces with the first attachment location 1008 being arranged in a first piece 1014 of the base 1002 and the second attachment location 1010 being arranged in a second piece 1016 of the baes 1002. After splitting, the first anchor assembly 1004 and the first piece 1014 of the base 1002 and the second anchor assembly 1006 and the second piece 1016 of the base 1002 move with an attached leaflet 20, 22 and separately from each other.

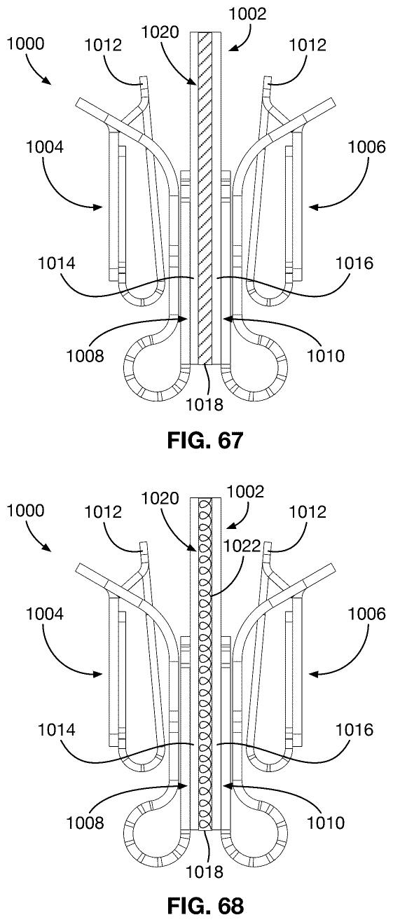

In some implementations, to achieve splitting the base 1002 into first and second pieces 1014, 1016, the base 1002 is formed from a suitable material, such as a polymer material that is at least partially embedded with a radiofrequency-reactive material 1020 along a center portion 1018. Referring now to FIGS. 67-68, different forms of the radiofrequency-reactive material 1020 in the base 1002 are shown.

Each of the arrangements of the base 1002 shown in FIGS. 67-68 is configured to separate the base 1002 into first and second pieces 1014, 1016. In some implementations, the base 1002 in FIG. 67 includes a radiofrequency-reactive material 1020 embedded throughout the center portion 1018 base 1002 so that exposure to a radiofrequency field increases the temperature of the center portion 1018 so that the material of the center portion 1018 melts, thereby separating the base 1002 into first and second pieces 1014, 1016 each including a respective anchor assembly 1004, 1006. In the example of FIG. 68, the radiofrequency-reactive material 1020 embedded in the center portion 1018 is shown as an antenna 1022 that increases in temperature when exposed to a radiofrequency field. The antenna 1022 can be tuned to respond to a particular radiofrequency or a range of radiofrequencies. The increased temperature of the antenna 1022 after exposure to a radiofrequency field causes the material of the center portion 1018 of the base 1002 to melt to separate the base 1002 into first and second pieces 1014, 1016 each including a respective anchor assembly 1004, 1006.

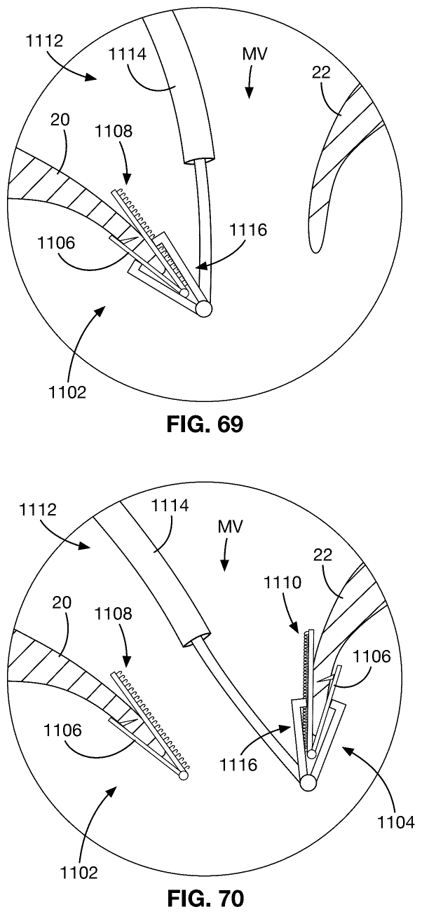

Referring now to FIGS. 69-74, an implantable valve treatment device 1100 is shown. The device 1100 can include any of the features of other valve treatment devices described herein. In some implementations, the device 1100 includes a first anchor assembly 1102 and a second anchor assembly 1104. In some implementations, each of the first and second anchor assemblies 1102, 1104 includes a gripping member 1106 for securing the respective anchor assembly 1102, 1104 to one of the native leaflets 20, 22.

In some implementations, the first anchor assembly 1102 includes a first connecting member 1108 and the second anchor assembly 1104 includes a second connecting member 1110. In some implementations, the first and second connecting members 1108, 1110 facilitate the creation of a connection between the first and second anchor assemblies 1102, 1104 after the first and second anchor assemblies 1102, 1104 are attached to the native leaflets 20, 22, respectively.

The first and second connecting members 1108, 1110 can be formed from any suitable material or mechanism that enables the formation of the connection between the first and second anchor assemblies 1102, 1104 after each of the first and second anchor assemblies 1102, 1104 have each been attached to one of the native leaflets 20, 22. For example, in the illustrated example, the first connecting member 1108 is formed from a hook side of a hook-and-loop fastener and the second connecting member 1110 is formed from a loop side of a hook-and-loop fastener. In another example, each of the connecting members 1108, 1110 includes both the hook side and loop side of the hook-and-loop fastener.

Referring now to FIGS. 69-73, the steps of attaching the device 1100 to the native leaflets 20, 22 is shown. Referring now to FIGS. 69-70, each of the first and second anchor assemblies 1102, 1104 are attached separately to the native leaflets 20, 22. In particular, FIG. 69 shows the first anchor assembly 1102 being delivered by a delivery system 1112 into the native valve and attached to one of the native leaflets 20. In the example of FIG. 70, the second anchor assembly 1104 is delivered by the delivery system 1112 into the native valve and attached to the other of the native leaflets 22.

The delivery system 1112 can be any suitable delivery system, such as the delivery systems described herein. In some implementations, the first anchor assembly 1104 can be delivered via a catheter 1114 and secured in place via a tool 1116 that squeezes the gripping member 1106 onto the leaflet 20, 22. In some implementations, the gripping member 1106 can be biased in a closing direction and held open by, for example, an actuation member of the delivery system 1112.

In some implementations, the actuation member can be used to open the gripping member 1106 and position the gripping member 1106 so that allowing the gripping member 1106 to close by releasing the actuation member causes the leaflet 20, 22 to be captured by the gripping member 1106 to secure the anchor assembly 1102, 1104 to the leaflet 20, 22.

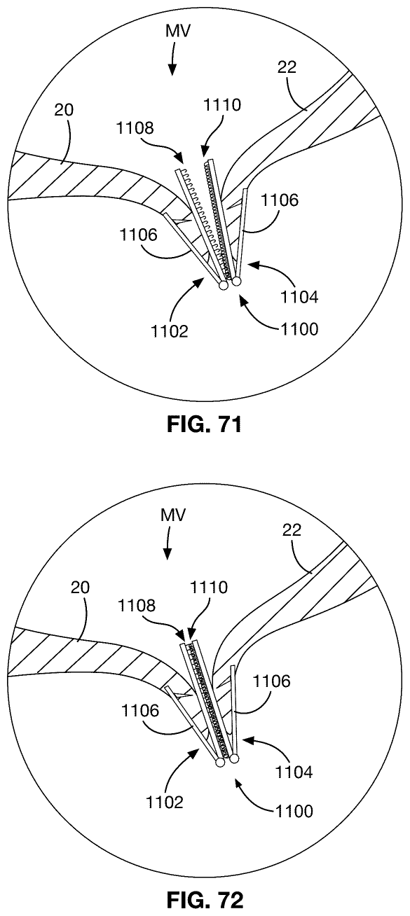

In some implementations, after implantation of the first and second anchor assemblies 1102, 1104, closing of the leaflets 20, 22 during systole causes the first connecting member 1108 to contact the second connecting member 1110 so that the hook-and-loop fasteners can form an initial connection between the first anchor assembly 1102 and the second anchor assembly 1104 as can be seen in FIG. 71. In some implementations, repeated closures of the native valve during subsequent beats of the heart cause the first and second connecting members 1108, 1110 to engage further until the first anchor assembly 1102 is mostly or fully connected to the second anchor assembly 1104 as shown in FIG. 72.

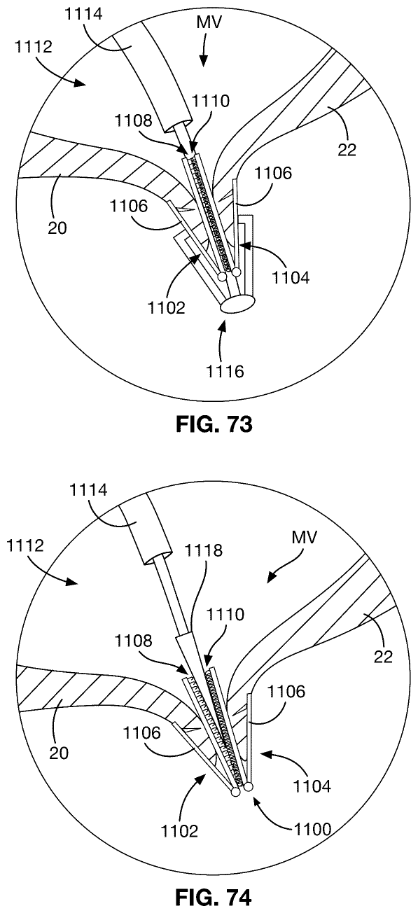

Referring now to FIG. 73, an optional installation tool 1116 can be delivered to the ventricular side of the native valve to further compress the first and second anchor assemblies 1102, 1104 together to ensure that the first and second connecting members 1108, 1110 are fully engaged. (In some implementations, the installation tool 1116 is inserted through an opening in the native valve be able to reach the device 1100 from the ventricular side of the valve.)

In some implementations, the connecting members 1108, 1110 can include bioabsorbable portions that eventually decompose and are replaced by tissue ingrowth.

In some implementations, the device 1110 can be separated after implantation to allow for the delivery and implantation of a new valve treatment device or a replacement valve. In some implementations, to facilitate the separation of the first and second anchor assemblies 1102, 1104, a separation tool 1118 can be deployed by the delivery system 1112 to disconnect the first connecting member 1108 from the second connecting member 1110. The separation tool 1118 can have any suitable shape, such as, for example, the wedge shape shown in FIG. 74.

### EXAMPLES

Example 1. A valve treatment device, comprising:
a coaptation element;
a first anchor assembly connected to a first side of the coaptation element, the first anchor assembly configured to grasp a first leaflet of a native heart valve;
a second anchor assembly connected to a second side of the coaptation element, the second anchor assembly configured to grasp a second leaflet of the native heart valve; and
a bioabsorbable central portion between the first anchor assembly and the second anchor assembly, wherein the bioabsorbable central portion includes the coaptation element.

Example 2. The valve treatment device of example 1, wherein the bioabsorbable central portion further comprises a first alignment member associated with the first anchor assembly and a second alignment member associated with the second anchor assembly.

Example 3. The valve treatment device of example 2, wherein the bioabsorbable central portion further comprises a coupling element configured to connect the coaptation element, the first anchor assembly, and the second anchor assembly together.

Example 4. The valve treatment device of example 3, wherein the coupling element threadedly engages at least one of the first alignment member and the second alignment member.

Example 5. The valve treatment device of example 3 or 4, wherein the coupling element extends through a first longitudinal passage in the coaptation element, a second longitudinal passage in the first alignment member, and into a third longitudinal passage in the second alignment member.

Example 6. The valve treatment device of any of examples 2-5, wherein the first anchor assembly includes a first inner paddle, and the second anchor assembly includes a second inner paddle, wherein the first alignment member is attached to the first inner paddle and the second alignment member is attached to the second inner paddle.

Example 7. The valve treatment device of example 6, wherein in an assembled condition, the first alignment member engages the second inner paddle, and the second alignment member engages the first inner paddle.

Example 8. The valve treatment device of any of examples 2-7, wherein the first alignment member and the second alignment member are each formed as rectangular blocks.

Example 9. The valve treatment device of any of examples 1-7, wherein the coaptation element comprises a plurality of lateral passages used to route actuation lines connected to the first anchor assembly and the second anchor assembly during deployment of the valve treatment device.

Example 10. The valve treatment device of example 9, wherein the plurality of lateral passages includes a first pair of lateral passages for routing a first actuation line connecting the first anchor assembly to the coaptation element and a second pair of lateral passages of routing a second actuation line connecting the first anchor assembly to the coaptation element.

Example 11. The valve treatment device of example 10, wherein the plurality of lateral passages includes a third pair of lateral passages for routing a third actuation line connecting the second anchor assembly to the coaptation element and a fourth pair of lateral passages of routing a fourth actuation line connecting the second anchor assembly to the coaptation element.

Example 12. The valve treatment device of any of examples 1-11, wherein the first anchor assembly comprises an inner paddle and an outer paddle connected to the inner paddle by a bendable portion, wherein the outer paddle is moveable between an open condition and a closed condition relative to the inner paddle.

Example 13. The valve treatment device of example 12, wherein the first anchor assembly further comprises a gripping member having a fixed arm attached to the outer paddle and a moveable arm connected to the fixed arm by a joint portion, wherein the moveable arm is moveable between an open condition and a closed condition relative to the fixed arm.

Example 14. The valve treatment device of any of examples 1-13, wherein the first anchor assembly is actuatable between an open condition and a closed condition independent of the second anchor assembly.

Example 15. The valve treatment device of any of examples 1-14, wherein during deployment of the valve treatment device, the first anchor assembly is connected to the coaptation element by a plurality of actuation lines.

Example 16. The valve treatment device of example 15, wherein during deployment of the valve treatment device, the first anchor assembly is selectively engageable and disengageable with the coaptation element while remaining connected to the coaptation element by the plurality of actuation lines.

Example 17. The valve treatment device of example 16, wherein when the first anchor assembly is disengaged from the coaptation element, the coaptation element and the second anchor assembly are movable relative to the first anchor assembly.

Example 18. A method of repairing a native heart valve, comprising:
connecting a first anchor assembly to a coaptation element by a first actuation line;
connecting a second anchor assembly to the coaptation element by second actuation line;
positioning a valve treatment device at the native heart valve;
attaching the first anchor assembly of the valve treatment device to a first valve leaflet of the native heart valve;
attaching the second anchor assembly of the valve treatment device to a second valve leaflet of the native heart valve;
attaching the first anchor assembly and the second anchor assembly to the coaptation element by a coupling element; and
removing the first actuation line and the second actuation line.

Example 19. The method of example 18, wherein positioning the valve treatment device at the native heart valve further comprising delivering the valve treatment device through a lumen of a catheter where the first anchor assembly, the second anchor assembly, and the coaptation element are axially aligned within the lumen.

Example 20. The method of example 18 or 19, wherein connecting the first anchor assembly to the coaptation element by the first actuation line further comprises routing the first actuation line through a first pair of lateral passages in the coaptation element.

Example 21. The method of example 20, wherein connecting the second anchor assembly to the coaptation element by the second actuation line further comprises routing the second actuation line through a second pair of lateral passages in the coaptation element.

Example 22. The method of example 20 or 21, wherein connecting the first anchor assembly to the coaptation element further comprises connecting the first anchor assembly to the coaptation element by a third actuation line and routing the third actuation line through a third pair of lateral passages in the coaptation element.

Example 23. The method of example 21, wherein connecting the second anchor assembly to the coaptation element further comprises connecting the second anchor assembly to the coaptation element by a fourth actuation line and routing the fourth actuation line through a fourth pair of lateral passages in the coaptation element.

Example 24. The method of any of example 18-23, further comprising tensioning the first actuation line to draw the first anchor assembly into engagement with the coaptation element.

Example 25. The method of example 24, further comprising tensioning the second actuation line to draw the second anchor assembly into engagement with the coaptation element.

Example 26. The method of example 25, wherein attaching the first anchor assembly and the second anchor assembly to the coaptation element by the coupling element further comprises receiving the coupling element in a first longitudinal passage in the coaptation element, a second longitudinal passage associated with the first anchor assembly, and a third longitudinal passage associated with the second anchor assembly.

Example 27. The method of example 26, further comprising threadedly engaging the coupling element with at least one of the second longitudinal passage and the third longitudinal passage.

Example 28. The method of any of examples 18-27, wherein attaching the first anchor assembly to the first valve leaflet is completed with the first anchor assembly disengaged from the coaptation element.

Example 29. The method of example 28, wherein attaching the second anchor assembly of the valve treatment device to the second valve leaflet further comprises engaging the coaptation element with the second anchor assembly and moving the coaptation element to move the second anchor assembly in position to attach to the second valve leaflet.

Example 30. The method of any of examples 18-29, wherein the coaptation element and the coupling element are bioabsorbable.

Example 31. A valve treatment system, comprising:
a delivery system comprising a catheter and a control handle;
a valve treatment device coupled to the delivery system, the valve treatment device comprising:
a coaptation element;
a first anchor assembly connected to a first side of the coaptation element, the first anchor assembly configured to grasp a first leaflet of a native heart valve;
a second anchor assembly connected to a second side of the coaptation element, the second anchor assembly configured to grasp a second leaflet of the native heart valve; and
a bioabsorbable central portion between the first anchor assembly and the second anchor assembly, the bioabsorbable central portion including the coaptation element.

Example 32. The valve treatment system of example 31, wherein the bioabsorbable central portion further comprises a first alignment member associated with the first anchor assembly and a second alignment member associated with the second anchor assembly.

Example 33. The valve treatment system of example 32, wherein the bioabsorbable central portion further comprises a coupling element configured to connect the coaptation element, the first anchor assembly, and the second anchor assembly together.

Example 34. The valve treatment system of example 33, wherein the coupling element threadedly engages at least one of the first alignment member and the second alignment member.

Example 35. The valve treatment system of example 33 or 34, wherein the coupling element extends through a first longitudinal passage in the coaptation element, a second longitudinal passage in the first alignment member, and into a third longitudinal passage in the second alignment member.

Example 36. The valve treatment system of any of examples 32-35, wherein the first anchor assembly includes a first inner paddle, and the second anchor assembly includes a second inner paddle, wherein the first alignment member is attached to the first inner paddle and the second alignment member is attached to the second inner paddle.

Example 37. The valve treatment system of example 36, wherein in an assembled condition, the first alignment member engages the second inner paddle, and the second alignment member engages the first inner paddle.

Example 38. The valve treatment system of any of examples 32-37, wherein the first alignment member and the second alignment member are each formed as rectangular blocks.

Example 39. The valve treatment system of any of examples 31-37, wherein the coaptation element comprises a plurality of lateral passages used to route actuation lines connected to the first anchor assembly and the second anchor assembly during deployment of the valve treatment device.

Example 40. The valve treatment system of example 39, wherein the plurality of lateral passages includes a first pair of lateral passages for routing a first actuation line connecting the first anchor assembly to the coaptation element and a second pair of lateral passages of routing a second actuation line connecting the first anchor assembly to the coaptation element.

Example 41. The valve treatment system of example 40, wherein the plurality of lateral passages includes a third pair of lateral passages for routing a third actuation line connecting the second anchor assembly to the coaptation element and a fourth pair of lateral passages of routing a fourth actuation line connecting the second anchor assembly to the coaptation element.

Example 42. The valve treatment system of any of examples 31-41, wherein the first anchor assembly comprises an inner paddle and an outer paddle connected to the inner paddle by a bendable portion, wherein the outer paddle is moveable between an open condition and a closed condition relative to the inner paddle.

Example 43. The valve treatment system of example 42, wherein the first anchor assembly further comprises a gripping member having a fixed arm attached to the outer paddle and a moveable arm connected to the fixed arm by a joint portion, wherein the moveable arm is moveable between an open condition and a closed condition relative to the fixed arm.

Example 44. The valve treatment device of any of examples 31-43, wherein the first anchor assembly is actuatable between an open condition and a closed condition independent of the second anchor assembly.

Example 45. The valve treatment device of any of examples 31-44, wherein during deployment of the valve treatment device, the first anchor assembly is connected to the coaptation element by a plurality of actuation lines.

Example 46. The valve treatment device of example 45, wherein during deployment of the valve treatment device, the first anchor assembly can selectively be engaged with the coaptation element and disengage with the coaptation element while remaining connected to the coaptation element by the plurality of actuation lines.

Example 47. The valve treatment device of example 46, wherein when the first anchor assembly is disengaged from the coaptation element, the coaptation element and the second anchor assembly are movable relative to the first anchor assembly.

Example 48. A valve treatment device, comprising:
a base;
a first anchor assembly connected to a first side of the base, the first anchor assembly configured to grasp a first leaflet of a native heart valve;
a second anchor assembly connected to a second side of the base, the second anchor assembly configured to grasp a second leaflet of the native heart valve; and
a connecting member comprising a radiofrequency-reactive material, wherein the connecting member connects at least the second anchor assembly to the base.

Example 49. The valve treatment device of example 48, wherein the base further comprises a coaptation member.

Example 50. The valve treatment device of example 48 or example 49, wherein the connecting member is a polymer suture embedded with the radiofrequency-reactive material.

Example 51. The valve treatment device of any of examples 48-50, wherein the base and the connecting member are formed as a single component.

Example 52. The valve treatment device of example 51, wherein the base is formed from a polymer material and the radiofrequency-reactive material is embedded in a connection portion of the base that is connected to the second anchor assembly.

Example 53. The valve treatment device of any of examples 48-52, wherein the radiofrequency-reactive material is embedded in a center portion of the base.

Example 54. The valve treatment device of any of examples 48-52, wherein the radiofrequency-reactive material is an antenna.

Example 55. A method of repairing a native heart valve, comprising:
locating a valve treatment device attached to the native heart valve, the valve treatment device comprising a first anchor assembly connected to a first side of a base and to a first valve leaflet of the native heart valve, a second anchor assembly connected to a second side of the base and to a second valve leaflet of the native heart valve, and a connecting member connecting at least the second anchor assembly to the base, wherein the connecting member comprises a radiofrequency-reactive material;
disconnecting the second anchor assembly from the base by directing radiofrequency radiation toward the connecting member; and
positioning a replacement valve between the first valve leaflet and the second valve leaflet of the native heart valve.

Example 56. The method of example 55, wherein the base further comprises a coaptation member.

Example 57. The method of example 55 or example 56, wherein the connecting member is a polymer suture embedded with the radiofrequency-reactive material.

Example 58. The method of any of examples 55-57, wherein the base and the connecting member are formed as a single component.

Example 59. The method of any of examples 55-58, wherein the base is formed from a polymer material and the radiofrequency-reactive material is embedded in a connection portion of the base that is connected to the second anchor assembly.

Example 60. The method of any of examples 55-59, wherein the radiofrequency-reactive material is embedded in a center portion of the base.

Example 61. The method of any of examples 55-60, wherein the radiofrequency-reactive material is an antenna.

Example 62. A valve treatment device, comprising:
a first anchor assembly configured to grasp a first leaflet of a native heart valve, wherein the first anchor assembly comprises a first connecting member;
a second anchor assembly configured to grasp a second leaflet of the native heart valve, wherein the second anchor assembly comprises a second connecting member; and

wherein the first connecting member comprises a hook portion of a hook-and-loop fastener and the second connecting member comprises a loop portion of the hook-and-loop fastener.

Example 63. The valve treatment device of example 62, wherein the first connecting member comprises a loop portion of the hook-and-loop fastener and the second connecting member comprises a hook portion of the hook-and-loop fastener.

Example 64. The valve treatment device of any of example 62 or example 63, wherein the first and second connecting members comprise a bioabsorbable material.

Example 65. A method of repairing a native heart valve, comprising:
attaching a first anchor assembly to a first leaflet of the native heart valve, wherein the first anchor assembly comprises a first connecting member, the first connecting member comprising a hook portion of a hook-and-loop fastener;
attaching a second anchor assembly to a second leaflet of the native heart valve, wherein the second anchor assembly comprises a second connecting member, the second connecting member comprising a loop portion of the hook-and-loop fastener;
separating the first anchor assembly from the second anchor assembly with a separation tool; and
positioning a replacement valve between the first valve leaflet and the second valve leaflet of the native heart valve.

Example 66. The method of example 65, further comprising a step of compressing the first anchor assembly against the second anchor assembly with an installation tool.

Example 67. The method of example 65 or example 66, wherein the first connecting member comprises a loop portion of the hook-and-loop fastener and the second connecting member comprises a hook portion of the hook-and-loop fastener.

Example 68. The method of any of examples 65-67, wherein the first and second connecting members comprise a bioabsorbable material.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A valve treatment device, comprising:
a bioabsorbable central portion comprising a coaptation element;
a first anchor assembly connected to a first side of the coaptation element, the first anchor assembly configured to grasp a first leaflet of a native heart valve;
a second anchor assembly connected to a second side of the coaptation element, the second anchor assembly configured to grasp a second leaflet of the native heart valve; and
wherein the bioabsorbable central portion further comprises a first alignment member associated with the first anchor assembly and a second alignment member associated with the second anchor assembly.

2. The valve treatment device of claim 1, wherein the bioabsorbable central portion further comprises a coupling element configured to connect the coaptation element, the first anchor assembly, and the second anchor assembly together.

3. The valve treatment device of claim 2, wherein the coupling element threadedly engages at least one of the first alignment member and the second alignment member.

4. The valve treatment device of claim 2, wherein the coupling element extends through a first longitudinal passage in the coaptation element, a second longitudinal passage in the first alignment member, and into a third longitudinal passage in the second alignment member.

5. The valve treatment device of claim 1, wherein the coaptation element comprises a plurality of lateral passages used to route actuation lines connected to the first anchor assembly and the second anchor assembly during deployment of the valve treatment device.

6. The valve treatment device of claim 5, wherein the plurality of lateral passages includes a first pair of lateral passages for routing a first actuation line connecting the first anchor assembly to the coaptation element and a second pair of lateral passages of routing a second actuation line connecting the first anchor assembly to the coaptation element.

7. The valve treatment device of claim 6, wherein the plurality of lateral passages includes a third pair of lateral passages for routing a third actuation line connecting the second anchor assembly to the coaptation element and a fourth pair of lateral passages of routing a fourth actuation line connecting the second anchor assembly to the coaptation element.

8. The valve treatment device of claim 1, wherein the first anchor assembly comprises an inner paddle and an outer paddle connected to the inner paddle by a bendable portion, wherein the outer paddle is moveable between an open condition and a closed condition relative to the inner paddle.

9. The valve treatment device of claim 8, wherein the first anchor assembly further comprises a gripping member having a fixed arm attached to the outer paddle and a moveable arm connected to the fixed arm by a joint portion, wherein the moveable arm is moveable between an open condition and a closed condition relative to the fixed arm.

10. A valve treatment device, comprising:
a base;
a first anchor assembly connected to a first side of the base, the first anchor assembly configured to grasp a first leaflet of a native heart valve;
a second anchor assembly connected to a second side of the base, the second anchor assembly configured to grasp a second leaflet of the native heart valve; and
a connecting member comprising a radiofrequency-reactive material, wherein the connecting member connects at least the second anchor assembly to the base.

11. The valve treatment device of claim 10, wherein the base further comprises a coaptation member.

12. The valve treatment device of claim 10, wherein the connecting member is a polymer suture embedded with the radiofrequency-reactive material.

13. The valve treatment device of claim 10, wherein the base and the connecting member are formed as a single component.

14. The valve treatment device of claim 13, wherein the base is formed from a polymer material and the radiofrequency-reactive material is embedded in a connection portion of the base that is connected to the second anchor assembly.

15. The valve treatment device of claim 10, wherein the radiofrequency-reactive material is embedded in a center portion of the base.

16. The valve treatment device of claim 10, wherein the radiofrequency-reactive material is an antenna.

17. A valve treatment device, comprising:
a first anchor assembly configured to grasp a first leaflet of a native heart valve, wherein the first anchor assembly comprises a first connecting member;
a second anchor assembly configured to grasp a second leaflet of the native heart valve, wherein the second anchor assembly comprises a second connecting member; and
wherein the first connecting member comprises a hook portion of a hook-and-loop fastener and the second connecting member comprises a loop portion of the hook-and-loop fastener.

18. The valve treatment device of claim 17, wherein the first connecting member comprises a loop portion of the hook-and-loop fastener and the second connecting member comprises a hook portion of the hook-and-loop fastener.

19. The valve treatment device of claim 17, wherein the first and second connecting members comprise a bioabsorbable material.
